(19) United States
(12) Patent Application Publication
(10) Pub. No.: US 2022/0137067 A1
(43) Pub. Date: May 5, 2022

(54) ANTIBODY POTENCY ASSAY

(71) Applicant: Genentech, Inc., South San Francisco, CA (US)

(72) Inventors: Alexis DeHaven DUNKLE, San Carlos, CA (US); Jeongsup SHIM, Palo Alto, CA (US); Sahim Xavier WALLACE, Oakland, CA (US); Linda Git-Mon CHAN, San Mateo, CA (US); Catherine CRUZ, Mountain View, CA (US); Michael Thomas EBY, Burlingame, CA (US)

(73) Assignee: Genentech, Inc., South San Francisco, CA (US)

(21) Appl. No.: 17/503,122

(22) Filed: Oct. 15, 2021

Related U.S. Application Data
(63) Continuation of application No. PCT/US2020/028780, filed on Apr. 17, 2020.
(60) Provisional application No. 62/835,960, filed on Apr. 18, 2019.

(51) Int. Cl. G01N 33/68; G01N 33/50

(52) U.S. Cl. CPC G01N 33/6854; G01N 33/5023

(57) ABSTRACT

The present invention provides a cell-based assay for measuring antibody potency. Antigen, bound to a surface, is contacted with the antibody which in turn is contacted with a reporter cell. Compositions and kits are also contemplated.

### CROSS-REFERENCE TO RELATED APPLICATION

This application claims the benefit of U.S. Provisional Application 62/835,960, filed Apr. 18, 2019, the contents of which are hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 146392046740SeqList.txt, date recorded: Apr. 17, 2020, size: 1,112 bytes).

### FIELD OF THE INVENTION

The present invention provides methods for analyzing the potency of a polypeptide (e.g., an antibody or immunoadhesin). Compositions and kits are also contemplated.

### BACKGROUND OF THE INVENTION

Optimal antibody potency assays should be accurate, precise, and user-friendly, with short turnaround time and suitability for automation and high-throughput scaling. Several traditional bioassays to reflect ADCP and related mechanisms of action are available, such as PBMC-based methods, FACS-based methods, and ELISA for secreted cytokines. Unfortunately, many of these assays yield highly variable results and/or are time consuming. The novel potency assays described herein use a cell-based approach with reporter cells reflecting ADCP activity and can be used to detect antibody-antigen binding interactions.

All references cited herein, including patent applications and publications, are incorporated by reference in their entirety.

### BRIEF SUMMARY OF THE INVENTION

In some aspects, the invention provides a method for determining the activity of a polypeptide wherein the polypeptide binds a target antigen and the polypeptide comprises an Fc receptor binding domain, the method comprising a) contacting an immobilized target antigen with the polypeptide preparation to form an antigen-polypeptide complex, b) contacting the antigen-polypeptide complex with a phagocytic cell, wherein the phagocytic cell comprises an Fcγ receptor and nucleic acid encoding a reporter operably linked to a response element that is responsive to activation by the Fcγ receptor; wherein expression of the reporter indicates activity of the polypeptide.

In some aspects, the invention provides a method for quantitating the potency of a polypeptide preparation wherein the polypeptide binds a target antigen, the method comprising a) contacting a plurality of populations of immobilized target antigen with different concentrations of the polypeptide preparation to form antigen-polypeptide complexes, b) contacting the antigen-polypeptide complexes with a phagocytic cell, wherein the phagocytic cell comprises an Fcγ receptor and nucleic acid encoding a reporter operably linked to a response element that is responsive to activation by the Fcγ receptor, c) measuring expression of reporter, and d) determining the EC₅₀ of the polypeptide preparation and comparing the EC₅₀ of the polypeptide preparation with the EC₅₀ of a reference standard of the polypeptide of known potency. In some embodiments, the method further comprises calculating the potency based on the EC50 of the polypeptide preparation using a multi-parameter logistic fit against the reference standard. In some embodiments, the multi-parameter logistic fit is a 3-parameter, 4-parameter, or 5-parameter logistic fit. In some embodiments, the EC₅₀ of the reference standard is determined at the same time as the EC₅₀ of the polypeptide preparation.

In some embodiments of the above aspects, the reporter is a luciferase or a fluorescent protein. In some embodiments, the luciferase is a firefly luciferase, a Renilla luciferase, or a nanoluciferase. In some embodiments, the response element that is responsive to activation by the Fcγ receptor is an NFκB response element, an NFAT response element, an AP-1 response element, or an ERK-responsive transcription factor (e.g. Elk1).

In some embodiments of the above aspects; the phagocytic cell is a monocyte. In some embodiments, the phagocytic cell is from a cell line. In some embodiments, the cell line is a THP-1 cell line or a U-937 cell line. In some embodiments, the Fcγ receptor is a FcγRT (CD64) or FcγRIIa (CD32a) or FcγRIII (CD16). In some embodiments, the phagocytic cell is engineered to overexpress a Fcγ receptor. In some embodiments, the phagocytic cell is engineered to overexpress a FcγRIIa. In some embodiments, the phagocytic cell does not express FcγRIII.

In some embodiments of the above aspects, the target antigen is beta-amyloid (Aβ) or CD20. In some embodiments, the target antigen is beta-amyloid (Aβ). In some embodiments, the Aβ is human Aβ. In some embodiments, the Aβ comprises monomeric and/or oligomeric Aβ. In some embodiments, the human Aβ is Aβ 1-40 or Aβ 1-42. In some embodiments, the polypeptide comprises a full length Fc domain or an FcR-binding fragment of an Fe domain. In some embodiments, the polypeptide specifically binds Aβ. In some embodiments, the polypeptide is an antibody or an immunoadhesin. In some embodiments, the polypeptide in crenezumab.

In some embodiments of the above aspects, the target antigen is immobilized on a surface. In some embodiments, the surface is a plate. In some embodiments, the plate is a multi-well plate. In some embodiments, the antigen is immobilized to the surface at or near its N-terminus, at or near its C-terminus, or at or near its N-terminus and at or near its C-terminus. In some embodiments, the target antigen is immobilized on the surface using a biotin-streptavidin system. In some embodiments, the target antigen is bound to biotin and the surface comprises bound streptavidin. In some embodiments, the target antigen is bound to biotin at or near its N-terminus, at or near its C-terminus, or at or near its N-terminus and its C-terminus.

In some embodiments of the above aspects, the reporter is detected after about any one or more of 1, 2, 3, 4, 5, 6, 7, 8, 12, 16, 20, 24 hours or greater than 24 hours after contacting the antigen-polypeptide complex with the phagocytic cell.

In some aspects, the invention provides a kit for determining the potency of a polypeptide preparation wherein the polypeptide binds a target antigen and comprises an Fc receptor binding domain, the kit comprising an immobilized target antigen and a phagocytic cell, wherein the phagocytic cell comprises an Fcγ receptor and nucleic acid encoding a reporter operably linked to a response element that is responsive to activation by the Fcγ receptor, wherein expression of the reporter indicates potency of the polypeptide.

In some aspects, the invention provides a kit for quantitating the potency of an polypeptide preparation wherein the polypeptide binds a target antigen and comprises an Fc receptor binding domain, the kit comprising an immobilized target antigen, a phagocytic cell, and a reference standard; wherein the phagocytic cell comprises an Fcγ receptor and nucleic acid encoding a reporter operably linked to a response element that is responsive to activation by the Fcγ receptor, wherein expression of the reporter indicates potency of the polypeptide; and wherein the reference standard comprises a preparation of the polypeptide of known potency.

In some embodiments of the kits, the reporter is a luciferase or a fluorescent protein. In some embodiments, the luciferase is a firefly luciferase, a Renilla luciferase, or a nanoluciferase. In some embodiments, the response element that is responsive to activation by the Fcγ receptor is an NFκB response element, an NFAT response element, an AP-1 response element, or an ERK-responsive transcription factor (e.g. Elk1).

In some embodiments of the kits, the phagocytic cell is a monocyte. In some embodiments, the phagocytic cell is from a cell line. In some embodiments, the cell line is a THP-1 cell line or a U-937 cell line. In some embodiments, the Fcγ receptor is a FcγRI (CD64) or FcγRIIa (CD32a) or FcγRIII (CD16). In some embodiments, the phagocytic cell is engineered to overexpress a Fcγ receptor. In some embodiments, the phagocytic cell is engineered to overexpress a FcγRIIa. In some embodiments, the phagocytic cell does not express FcγRIII.

In some embodiments of the kits, the target antigen is beta-amyloid (Aβ) or CD20. In some embodiments, the target antigen is beta-amyloid (Aβ). In some embodiments, the Aβ is human Aβ. In some embodiments, the Aβ comprises monomeric and/or oligomeric Aβ. In some embodiments, the human Aβ is Aβ 1-40 or Aβ 1-42. In some embodiments, the polypeptide comprises a full length Fc domain or an FcR-binding fragment of an Fc domain. In some embodiments, the polypeptide specifically binds Aβ. In some embodiments, the polypeptide is an antibody or an immunoadhesin. In some embodiments, the polypeptide in crenezumab.

In some embodiments of the kits, the target antigen is immobilized on a surface. In some embodiments, the surface is a plate. In some embodiments, the plate is a multi-well plate. In some embodiments, the antigen is immobilized to the surface at or near its N-terminus, at or near its C-terminus, or at or near its N-terminus and at or near its C-terminus. In some embodiments, the target antigen is immobilized on the surface using a biotin-streptavidin system. In some embodiments, the target antigen is bound to biotin and the surface comprises bound streptavidin. In some embodiments, the target antigen is bound to biotin at or near its N-terminus, at or near its C-terminus, or at or near its N-terminus and its C-terminus. In some embodiments, the target antigen is immobilized on the surface using a biotin-streptavidin system. In some embodiments, the target antigen is bound to biotin and the surface comprises bound streptavidin.

### BRIEF DESCRIPTION OF THE DRAWINGS

FIG. 1 is a map showing construction of the CD32A expression vector.

FIG. 2 is a map showing construction of the NF-κB-luciferase expression vector.

FIG. 3 shows FcγR expression on phagocytosis reporter cells. Expression of CD16, CD32, and CD64 on parental U-937 cells, U-937 phagocytosis reporter cells, and THP-1 phagocytosis reporter cells is shown. Shaded histograms are unstained cells (included for U-937 only), solid line is CD16/CD32/CD64, and dashed line is isotype control. U937 cells and THP-1 cells were examined on different days using different instruments.

FIGS. 4A-4C show evaluation of different formats to incorporate Aβ peptide. THP-1 phagocytosis reporter cells (THP-1) were screened for activity using crenezumab and different forms of Aβ and assay plates. FIG. 4A shows soluble, non-biotinylated Aβ incubated with crenezumab and THP-1 cells. FIG. 4B shows non-biotinylated Aβ adsorbed onto high-binding plates followed by incubation with the crenezumab dilution series, then cells. FIG. 4C shows high-binding plates with adsorbed Aβ peptide compared with streptavidin (SA) high-binding plates bound with Biotin-Aβ. SA high-binding plates without Aβ were used as a negative control. Different clones (“Line XXX”) were evaluated for FIG. 4A and FIG. 4B. FIG. 4C utilized THP-1 Line 416.

FIG. 5 is a schematic of the potency assay.

FIG. 6 shows a representative standard curve for crenezumab.

FIG. 7 shows ocrelizumab activity in the phagocytosis reporter cell assay. Presented is a representative standard curve showing the ability of ocrelizumab to activate U-937 phagocytosis reporter cells upon binding to CD20 peptide as measured by luciferase reporter gene expression.

FIG. 8 shows growth of THP-1 at different seeding densities. Cells were seeded based on a target 3-day culture and monitored using an Incucyte Zoom. Numbers represent seeding density×10⁵ cells/ml.

FIG. 9 shows a dose response of THP-1 clones to recombinant vs. synthetic Aβ. Endotoxin testing results of recombinant Aβ showed 912 EU/mg of bacterial lipopolysaccharide (LPS) whereas the synthetic peptide was below the limit of detection.

FIG. 10 shows factors that impact EC₅₀.

FIG. 11 shows factors that impact slope.

FIG. 12 shows factors that impact fold response.

FIG. 13 shows factors that impact potency (mean and standard deviation).

### DETAILED DESCRIPTION OF THE INVENTION

In some aspects, the invention provides methods for determining the activity of a polypeptide wherein the polypeptide binds a target antigen and the polypeptide comprises an Fc receptor binding domain, the method comprising a) contacting an immobilized target antigen with the polypeptide preparation to form an antigen-polypeptide complex, b) contacting the antigen-polypeptide complex with a phagocytic cell, wherein the phagocytic cell comprises an Fcγ receptor and nucleic acid encoding a reporter operably linked to a response element that is responsive to activation by the Fcγ receptor; wherein expression of the reporter indicates activity of the polypeptide. In some aspects, the invention provides methods for quantitating the potency of a polypeptide preparation wherein the polypeptide binds a target antigen, the method comprising a) contacting a plurality of populations of immobilized target antigen with different concentrations of the polypeptide preparation to form antigen-polypeptide complexes, b) contacting the antigen-polypeptide complexes with a phagocytic cell, wherein the phagocytic cell comprises an Fcγ receptor and nucleic acid encoding a reporter operably linked to a response element that is responsive to activation by the Fcγ receptor, c) measuring expression of reporter, and d) determining the EC₅₀ of the polypeptide preparation and comparing the EC₅₀ of the polypeptide preparation with the EC₅₀ of a reference standard of the polypeptide of known potency. In some embodiments, the polypeptide is an antibody or an immunoadhesin. Compositions and kits are also provided.

### Definitions

The term “polypeptide” or “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component or toxin. Also included within the definition are, for example, polypeptides containing one or more analogs of an amino acid (including, for example, unnatural amino acids, etc.), as well as other modifications known in the art. The terms “polypeptide” and “protein” as used herein specifically encompass antibodies.

“Purified” polypeptide (e.g., antibody or immunoadhesin) means that the polypeptide has been increased in purity, such that it exists in a form that is more pure than it exists in its natural environment and/or when initially synthesized and/or amplified under laboratory conditions. Purity is a relative term and does not necessarily mean absolute purity.

The term “antagonist” is used in the broadest sense, and includes any molecule that partially or fully blocks, inhibits, or neutralizes a biological activity of a native polypeptide. In a similar manner, the term “agonist” is used in the broadest sense and includes any molecule that mimics a biological activity of a native polypeptide. Suitable agonist or antagonist molecules specifically include agonist or antagonist antibodies or antibody fragments, fragments or amino acid sequence variants of native polypeptides, etc. Methods for identifying agonists or antagonists of a polypeptide may comprise contacting a polypeptide with a candidate agonist or antagonist molecule and measuring a detectable change in one or more biological activities normally associated with the polypeptide.

A polypeptide “which binds” an antigen of interest is one that binds the antigen with sufficient affinity such that the polypeptide is useful as a diagnostic and/or therapeutic agent in targeting a cell or tissue expressing the antigen, and does not significantly cross-react with other polypeptides. In such embodiments, the extent of binding of the polypeptide to a “non-target” polypeptide will be less than about 10% of the binding of the polypeptide to its particular target polypeptide as determined by fluorescence activated cell sorting (FACS) analysis or radioimmunoprecipitation (RIA).

With regard to the binding of a polypeptide to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target.

The term “antibody” herein is used in the broadest sense and specifically covers monoclonal antibodies, polyclonal antibodies, multispecific antibodies (e.g. bispecific antibodies including TDB) formed from at least two intact antibodies, and antibody fragments so long as they exhibit the desired biological activity. The term “immunoglobulin” (Ig) is used interchangeable with antibody herein.

Antibodies are naturally occurring immunoglobulin molecules which have varying structures, all based upon the immunoglobulin fold. For example, IgG antibodies have two “heavy” chains and two “light” chains that are disulphide-bonded to form a functional antibody. Each heavy and light chain itself comprises a “constant” (C) and a “variable” (V) region. The V regions determine the antigen binding specificity of the antibody, whilst the C regions provide structural support and function in non-antigen-specific interactions with immune effectors. The antigen binding specificity of an antibody or antigen-binding fragment of an antibody is the ability of an antibody to specifically bind to a particular antigen.

The antigen binding specificity of an antibody is determined by the structural characteristics of the V region. The variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” (HVRs) that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

Each V region typically comprises three HVRs, e.g. complementarity determining regions (“CDRs”, each of which contains a “hypervariable loop”), and four framework regions. An antibody binding site, the minimal structural unit required to bind with substantial affinity to a particular desired antigen, will therefore typically include the three CDRs, and at least three, preferably four, framework regions interspersed there between to hold and present the CDRs in the appropriate conformation. Classical four chain antibodies have antigen binding sites which are defined by V_{H} and V_{L} domains in cooperation. Certain antibodies, such as camel and shark antibodies, lack light chains and rely on binding sites formed by heavy chains only. Single domain engineered immunoglobulins can be prepared in which the binding sites are formed by heavy chains or light chains alone, in absence of cooperation between V_{H} and V_{L}.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” (HVR) when used herein refers to the amino acid residues of an antibody that are responsible for antigen binding. The hypervariable region may comprise amino acid residues from a “complementarity determining region” or “CDR” (e.g., around about residues 24-34 (L1), 50-56 (L2) and 89-97 (L3) in the V_{L}, and around about 31-35B (H1), 50-65 (H2) and 95-102 (H3) in the V_{H} (Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the V_{L}, and 26-32 (H1), 52A-55 (H2) and 96-101 (H3) in the V_{H} (Chothia and Lesk J. Mol. Biol. 196:901-917 (1987)).

“Framework” or “FR” residues are those variable domain residues other than the hypervariable region residues as herein defined.

“Antibody fragments” comprise a portion of an intact antibody, preferably comprising the antigen binding region thereof. Examples of antibody fragments include Fab, Fab′, F(ab′)2, and Fv fragments; diabodies; tandem diabodies (taDb), linear antibodies (e.g., U.S. Pat. No. 5,641,870, Example 2; Zapata et al., Protein Eng. 8(10):1057-1062 (1995)); one-armed antibodies, single variable domain antibodies, minibodies, single-chain antibody molecules; multispecific antibodies formed from antibody fragments (e.g., including but not limited to, db-Fc, taDb-Fc, taDb-CH3, (scFV)4-Fc, di-scFv, bi-scFv, or tandem (di,tri)-scFv); and Bi-specific T-cell engagers (BiTEs).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-binding sites and is still capable of cross-linking antigen.

“Fv” is the minimum antibody fragment that contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)2 antibody fragments originally were produced as pairs of Fab′ fragments that have hinge cysteines between them. Other chemical couplings of antibody fragments are also known.

The “light chains” of antibodies (immunoglobulins) from any vertebrate species can be assigned to one of two clearly distinct types, called kappa (lc) and lambda (i), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy chain constant domains that correspond to the different classes of antibodies are called α, δ, ε, γ, and μ, respectively. The subunit structures and three-dimensional configurations of different classes of immunoglobulins are well known.

“Single-chain Fv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains that enables the scFv to form the desired structure for antigen binding. For a review of scFv see Plückthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain; the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The term “multispecific antibody” is used in the broadest sense and specifically covers an antibody that has polyepitopic specificity. Such multispecific antibodies include, but are not limited to, an antibody comprising a heavy chain variable domain (V_{H}) and a light chain variable domain (V_{L}), where the V_{H}V_{L} unit has polyepitopic specificity, antibodies having two or more V_{L} and V_{H} domains with each V_{H}V_{L} unit binding to a different epitope, antibodies having two or more single variable domains with each single variable domain binding to a different epitope, full length antibodies, antibody fragments such as Fab, Fv, dsFv, scFv, diabodies, bispecific diabodies, triabodies, tri-functional antibodies, antibody fragments that have been linked covalently or non-covalently. “Polyepitopic specificity” refers to the ability to specifically bind to two or more different epitopes on the same or different target(s). “Monospecific” refers to the ability to bind only one epitope. According to one embodiment the multispecific antibody is an IgG antibody that binds to each epitope with an affinity of 5 μM to 0.001 pM, 3 μM to 0.001 pM, 1 μM to 0.001 pM, 0.5 μM to 0.001 pM, or 0.1 μM to 0.001 pM.

The expression “single domain antibodies” (sdAbs) or “single variable domain (SVD) antibodies” generally refers to antibodies in which a single variable domain (VH or VL) can confer antigen binding. In other words; the single variable domain does not need to interact with another variable domain in order to recognize the target antigen. Examples of single domain antibodies include those derived from camelids (lamas and camels) and cartilaginous fish (e.g., nurse sharks) and those derived from recombinant methods from humans and mouse antibodies (Nature (1989) 341:544-546; Dev Comp Immunol (2006) 30:43-56; Trend Biochem Sci (2001) 26:230-235; Trends Biotechnol (2003):21:484-490; WO 2005/035572; WO 03/035694; Febs Lett (1994) 339:285-290; WO00/29004; WO 02/051870).

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. In addition to their specificity, the monoclonal antibodies are advantageous in that they are uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the methods provided herein may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA 81:6851-6855 (1984)). Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences (U.S. Pat. No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence, except for FR substitution(s) as noted above. The humanized antibody optionally also will comprise at least a portion of an immunoglobulin constant region, typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

For the purposes herein, an “intact antibody” is one comprising heavy and light variable domains as well as an Fc region. The constant domains may be native sequence constant domains (e.g. human native sequence constant domains) or amino acid sequence variant thereof. Preferably, the intact antibody has one or more effector functions.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

A “naked antibody” is an antibody (as herein defined) that is not conjugated to a heterologous molecule, such as a cytotoxic moiety or radiolabel.

As used herein, the term “effector function” or “Fc-mediated effector function” refers to those biological activities attributable to the Fc region (a native sequence Fc region or amino acid sequence variant Fc region) of an antibody, and vary with the antibody isotype. Examples of antibody effector functions include, but are not limited to: C1q binding and complement dependent cytotoxicity (CDC), Fc receptor binding affinity, antibody-dependent cell-mediated cytotoxicity (ADCC), antibody-dependent cellular phagocytosis (ADCP), and cytokine secretion.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils; and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells in summarized is Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally; ADCC activity of the molecule of interest may be assessed in vivo, e.g., in an animal model such as that disclosed in Clynes et al., Proc. Natl. Acad. Sci. (USA) 95:652-656 (1998).

“Human effector cells” are leukocytes that express one or more FcRs and perform effector functions. In some embodiments, the cells express at least FcγRIII and carry out ADCC effector function. Examples of human leukocytes that mediate ADCC include peripheral blood mononuclear cells (PBMC), natural killer (NK) cells, monocytes, cytotoxic T cells and neutrophils; with PBMCs and NK cells being preferred.

“Complement dependent cytotoxicity” or “CDC” refers to the ability of a molecule to lyse a target in the presence of complement. The complement activation pathway is initiated by the binding of the first component of the complement system (C1q) to a molecule (e.g. polypeptide (e.g., an antibody)) complexed with a cognate antigen. To assess complement activation, a CDC assay, e.g. as described in Gazzano-Santoro et al., J. Immunol. Methods 202:163 (1996), may be performed.

The term “antibody-dependent cellular phagocytosis”, or “ADCP”, denotes a process by which antibody-coated cells are internalized, either in whole or in part, by phagocytic immune cells (e.g. macrophages, neutrophils, or dendritic cells) that bind to an immunoglobulin Fc-region.

The terms “Fc receptor” or “FcR” are used to describe a receptor that binds to the Fc region of an antibody. In some embodiments, the FcR is a native sequence human FcR. Moreover, a preferred FcR is one that binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

The term “Aβ(X-Y)” herein refers to the amino acid sequence from amino acid position X to amino acid position Y of the human amyloid β protein including. Both X and Y refer to the amino acid sequence from amino acid position X to amino acid position Y of the amino acid sequence DAEFRHDSGYEVHHQKLVFFAEDVGSNKGAIIGLMVGGVVIA (SEQ ID NO.:1) or any of its naturally occurring variants, in particular, those with at least one mutation selected from the group consisting of A2T, H6R, D7N, A21G (“Flemish”), E22G (“Arctic”), E22Q (“Dutch”), E22K (“Italian”), D23N (“Iowa”), A42T and A42V wherein the numbers are relative to the start position of the Aβ peptide, including both position X and position Y or a sequence with up to three additional amino acid substitutions none of which may prevent globulomer formation. An “additional” amino acid substitution is defined herein as any deviation from the canonical sequence that is not found in nature.

More specifically, the term “Aβ(1-42)” herein refers to the amino acid sequence from amino acid position 1 to amino acid position 42 of the human amyloid β protein including both 1 and 42 and, in particular, refers to the amino acid sequence from amino acid position 1 to amino acid position 42 of the amino acid sequence

(SEQ ID NO.: 1)
DAEFRHDSGYEVHHQKLVFFAEDVGSNKGAIIGLMVGGVVIA

(corresponding to amino acid positions 1 to 42) or any of its naturally occurring variants. Such variants may be, for example, those with at least one mutation selected from the group consisting of A2T, H6R, D7N, A21G (“Flemish”), E22G (“Arctic”), E22Q (“Dutch”), E22K (“Italian”), D23N (“Iowa”), A42T and A42V wherein the numbers are relative to the start of the Aβ peptide, including both amino acid position 1 and amino acid position 42 or a sequence with up to three additional amino acid substitutions none of which may prevent globulomer formation. Likewise, the term “Aβ(1-40)” herein refers to the amino acid sequence from amino acid position 1 to amino acid position 40 of the human amyloid protein including both amino acid position 1 and amino acid position 40 and refers, in particular, to the amino acid sequence from amino acid position 1 to amino acid position 40 of the amino acid sequence DAEFRHDSGYEVHHQKLVFFAEDVGSNKGAIIGLMVGGVV (SEQ ID NO.: 2) or any of its naturally occurring variants. Such variants include, for example, those with at least one mutation selected from the group consisting of A2T, H6R, D7N, A21G (“Flemish”), E22G (“Arctic”), E22Q (“Dutch”), E22K (“Italian”), and D23N (“Iowa”) wherein the numbers are relative to the start position of the Aβ peptide, including both amino acid position 1 and amino acid position 40 or a sequence with up to three additional amino acid substitutions none of which may prevent globulomer formation.

“Contaminants” refer to materials that are different from the desired polypeptide product. In some embodiments of the invention, contaminants include charge variants of the polypeptide. In some embodiments of the invention, contaminants include charge variants of an antibody or antibody fragment. In other embodiments of the invention, the contaminant includes, without limitation: host cell materials, such as CHOP; leached Protein A; nucleic acid; a variant, fragment, aggregate or derivative of the desired polypeptide; another polypeptide; endotoxin; viral contaminant; cell culture media component, etc. In some examples, the contaminant may be a host cell protein (HCP) from, for example but not limited to, a bacterial cell such as an E. coli cell, an insect cell, a prokaryotic cell, a eukaryotic cell, a yeast cell, a mammalian cell, an avian cell, a fungal cell.

As used herein, the term “immunoadhesin” designates antibody-like molecules which combine the binding specificity of a heterologous polypeptide with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG1, IgG2, IgG3, or IgG4 subtypes, IgA (including IgA1 and IgA2), IgE, IgD or IgM.

By “reporter molecule”, as used in the present specification, is meant a molecule which, by its chemical nature, provides an analytically identifiable signal which allows the detection of antibody activity. The most commonly used reporter molecules in this type of assay are either enzymes, fluorophores or radionuclide containing molecules (i.e. radioisotopes) and chemiluminescent molecules.

As used herein “essentially the same” indicates that a value or parameter has not been altered by a significant effect. For example, an ionic strength of a chromatography mobile phase at column exit is essentially the same as the initial ionic strength of the mobile phase if the ionic strength has not changed significantly. For example, an ionic strength at column exit that is within 10%, 5% or 1% of the initial ionic strength is essentially the same as the initial ionic strength.

Reference to “about” a value or parameter herein includes (and describes) variations that are directed to that value or parameter per se. For example, description referring to “about X” includes description of “X”.

As used herein and in the appended claims, the singular forms “a,” “or,” and “the” include plural referents unless the context clearly dictates otherwise. It is understood that aspects and variations of the invention described herein include “consisting” and/or “consisting essentially of” aspects and variations.

### Cell-Based Potency Assays

The present invention provides cell-based assays to determine or activity or potency of a polypeptide preparation wherein the polypeptide comprises an antigen binding domain and an Fc receptor binding domain. The antigen binding domain of the polypeptide binds to an immobilized antigen then is contacted with a phagocytic cell comprising an Fc receptor such that when the Fc receptor binds the Fc domain of the polypeptide, a reporter is activated. Activity of the reporter, which correlates with expression of the reporter, is then compared to activity of a reporter activated by a polypeptide of known activity or potency. In some embodiments, the polypeptide is an antibody or an immunoadhesin. The cell-based assays are useful, inter alia, for detecting the polypeptide in a composition, quantitating the amount of polypeptide in a composition, determining the specificity of the polypeptide in the composition and/or determining the potency of the polypeptide composition.

### Reporters

A reporter assay is an analytical method that enables the biological characterization of a stimulus by monitoring the induction of expression of a reporter in a cell. The stimulus leads to the induction of intracellular signaling pathways that result in a cellular response that typically includes modulation of gene transcription. In some examples, stimulation of cellular signaling pathways result in the modulation of gene expression via the regulation and recruitment of transcription factors to upstream non-coding regions of DNA that are required for initiation of RNA transcription leading to protein production. Control of gene transcription and translation in response to a stimulus is required to elicit the majority of biological responses such as cellular proliferation, differentiation, survival and immune responses. These non-coding regions of DNA, also called response elements, contain specific sequences that are the recognition elements for transcription factors which regulate the efficiency of gene transcription and thus, the amount and type of proteins generated by the cell in response to a stimulus. In a reporter assay, a response element and minimal promoter that is responsive to a stimulus is engineered to drive the expression of a reporter gene using standard molecular biology methods. The DNA is then transfected or transduced into a cell, which contains all the machinery to specifically respond to the stimulus, and the level of reporter gene transcription, translation, or activity is measured as a surrogate measure of the biological response.

In some aspects, the invention provides methods for determining the activity of an polypeptide preparation wherein the polypeptide binds a target antigen and comprises an Fc receptor binding domain (e.g., an Fcγ receptor binding domain), the method comprising a) contacting an immobilized target antigen with the polypeptide preparation to form an antigen-polypeptide complex, b) contacting the antigen-polypeptide complex with a phagocytic cell, wherein the phagocytic cell comprises an Fcγ receptor and nucleic acid encoding a reporter operably linked to a response element that is responsive to activation by the Fcγ receptor; wherein expression of the reporter indicates activity of the polypeptide. In some aspects, the invention provides methods for quantitating the potency of an polypeptide preparation wherein the polypeptide binds a target antigen and comprises an Fc receptor binding domain (e.g., an Fcγ receptor binding domain), the method comprising a) contacting a plurality of populations of immobilized target antigen with different concentrations of the polypeptide preparation to form antigen-polypeptide complexes, b) contacting the antigen-polypeptide complexes with a phagocytic cell, wherein the phagocytic cell comprises an Fcγ receptor and nucleic acid encoding a reporter operably linked to a response element that is responsive to activation by the Fcγ receptor, c) measuring expression of reporter, and d) determining the EC₅₀ of the polypeptide preparation and comparing the EC₅₀ of the polypeptide preparation with the EC₅₀ of a reference standard of the polypeptide of known potency. In some embodiments, the polypeptide is an antibody or an immunoadhesin. A reporter may be any molecule for which an assay can be developed to measure the amount of that molecule that is produced by the cell in response to the stimulus. For example, a reporter may be a reporter protein that is encoded by a reporter gene that is responsive to the stimulus (e.g., polypeptide binding to an Fc receptor). Commonly used examples of reporter molecules include, but are not limited to, luminescent proteins such as luciferase, which emit light that can be measured experimentally as a by-product of the catalysis of substrate. Luciferases are a class of luminescent proteins that are derived from many sources and include firefly luciferase (from the species, Photinus pyralis); Renilla luciferase from sea pansy (Renilla reniformis), click beetle luciferase (from Pyrearinus termitilluminans), marine copepod Gaussia luciferase (from Gaussia princeps), and deep sea shrimp Nano luciferase (from Oplophorus gracilirostris). Firefly luciferase catalyzes the oxygenation of luciferin to oxyluciferin, resulting in the emission of light, while other luciferases, such as Renilla, emit light by catalyzing the oxygenation of coelenterazine. The wavelength of light emitted by different luciferase forms and variants can be read using different filter systems, which facilitates multiplexing. The amount of luminescence is proportional to the amount of luciferase expressed in the cell, and luciferase genes have been used as a sensitive reporter to quantitatively evaluate the potency of a stimulus to elicit a biological response. Reporter gene assays have been used for many years for a wide range of purposes including basic research, HTS screening, and for potency (Brogan J, et al., 2012, Radiat Res. 177(4):508-513; Miraglia L J, et al., 2011, Comb Chem High Throughput Screen. 14(8):648-657; Nakajima Y, and Ohmiya Y. 2010, Expert Opin Drug Discovery, 5(9):835-849; Parekh B S, et al., 2012, Mabs, 4(3):310-318; Svobodova K, and Cajtham L T., 2010, Appl Microbiol Biotechnol., 88(4): 839-847).

In some embodiments, the invention provides cell-based assays to determine the activity and/or potency of a polypeptide where a polypeptide-antigen complex is contacted with an engineered phagocytic cell comprising a reporter complex. In some embodiments, the reporter construct comprises a luciferase. In some embodiments, the luciferase is a firefly luciferase (e.g., from the species Photinus pyralis), Renilla luciferase from sea pansy (e.g., from the species Renilla reniformis), click beetle luciferase (e.g., from the species Pyrearinus termitilluminans), marine copepod Gaussia luciferase (e.g., from the species Gaussia princeps), or deep sea shrimp Nano luciferase (e.g., from the species Oplophorus gracilirostris). In some embodiments, expression of luciferase in the engineered phagocytic cell indicates the binding activity of the polypeptide or immunoadhesin to the phagocytic cell. In other aspects, the reporter construct encodes a β-glucuronidase (GUS); a fluorescent protein such as green fluorescent protein (GFP), red fluorescent protein (RFP), blue fluorescent protein (BFP), yellow fluorescent protein (YFP) or variants thereof; a chloramphenicoal acetyltransferase (CAT); a β-galactosidase; a β-lactamase; or a secreted alkaline phosphatase (SEAP).

In some embodiments, there are provided engineered cells comprising nucleic acid encoding a reporter molecule (e.g., a reporter protein, such as a luciferase) operably linked to control sequences comprising a promoter and/or elements responsive to binding of an Fc domain to an Fc receptor on the surface of the cell. Promoter and/or element sequences can be selected from among any of those known in the art to be responsive to FcR activation. In some embodiments, the nucleic acid is stably integrated into the cell genome.

In some embodiments, there are provided engineered cells (e.g., phagocytic cells) comprising nucleic acid encoding a reporter molecule under the control of a minimal promoter operably linked to one or more FcR activation responsive elements. In some embodiments, the minimal promoter is a thymidine kinase (TK) minimal promoter, a minimal promoter from cytomegalovirus (CMV), an SV40-derived promoter, or a minimal elongation factor 1 alpha (EF1α) promoter. In some embodiments, the minimal promoter is a minimal TK promoter. In some embodiments, the minimal promoter is a minimal CMV promoter. In some embodiments, the activation responsive element comprises an NFAT (Nuclear Factor of Activated T cells) response element, AP-1 (Fos/Jun) response element, NFAT/AP1 response element, NFκB response element, FOXO response element, STAT3 response element, STAT5 response element or IRF response element. In some embodiments, the FcR activation responsive elements are arranged as tandem repeats (such as about any of 2, 3, 4, 5, 6, 7, 8, or more tandem repeats). The FcR activation responsive elements may be positioned 5′ or 3′ to the reporter-encoding sequence. In some embodiments, the FcR activation responsive elements are located at a site 5′ from the minimal promoter. In some embodiments, the FcR activation responsive elements are NFκB responsive elements. In some embodiments, the reporter molecule is a luciferase, such as firefly or Renilla luciferase. In some embodiments, the nucleic acid is stably integrated into the macrophage genome.

### Cells

In some embodiments, there are provided methods of determining the activity and/or potency of a polypeptide preparation wherein the polypeptide comprises an antigen-binding domain and an Fc receptor binding domain (e.g., an FcγR binding domain) by contacting a polypeptide-antigen complex with a population of cells comprising an Fcγ receptor and nucleic acid encoding a reporter operably linked to a response element that is responsive to activation by the Fcγ receptor. In some embodiments, the cell is a phagocytic cell. In some embodiments, the phagocytic cell is a monocyte. In some embodiments, the phagocytic cell is from a cell line. In some embodiments, the phagocytic cell line is a THP-1 cell line or a U-937 cell line.

In some embodiments, the reporter cell comprises an Fc receptor. In some embodiments, the Fc receptor is an Fcγ receptor. In some embodiments, the Fcγ receptor is an FcγRI (CD64), FcγRIIa (CD32a) and/or FcγRIII (CD16). In some embodiments, the reporter cell is engineered to express one or more of FcγRI (CD64), FcγRIIa (CD32a) or FcγRIII (CD16). In some embodiments, the reporter cell is engineered to overexpress one or more of FcγRI (CD64), FcγRIIa (CD32a) or FcγRIII (CD16). In some embodiments, the reporter cell is engineered to overexpress a FcγRIIa. In some embodiments, the reporter cell does not express FcγRIII.

In some embodiments, the reporter cells comprise nucleic acid encoding a reporter operably linked to a response element that is responsive to activation by an Fcγ receptor. In some embodiments, the reporter comprises a polynucleotide encoding a luciferase. In some embodiments, the luciferase is a firefly luciferase, a Renilla luciferase, or a nanoluciferase. In some embodiments, the polynucleotide encoding the reporter (e.g., luciferase) is operably linked to a FcR activation responsive regulatory element (e.g., an FcR activation responsive promoter and/or element). In some embodiments, the promoter and/or element responsive to FcR activation is an NFAT promoter, an AP-1 promoter, an NFκB promoter, a FOXO promoter, a STAT3 promoter, a STAT5 promoter or an IRF promoter. In some embodiments, the reporter cells comprise nucleic acid encoding a reporter operably linked to a response element that is responsive to activation by the Fcγ receptor and comprise one or more of an FcγRI, FcγRIIa or FcγRIII.

In some embodiments, the invention provides compositions of cells engineered with an FcR activation reporter construct encoding a reporter molecule operably linked to control sequences comprising a promoter and/or elements responsive to FcR activation. In some embodiments, the invention provides compositions of cells engineered with an FcγR activation reporter construct encoding a reporter molecule operably linked to control sequences comprising a promoter and/or elements responsive to FcγR activation. In some embodiments, the reporter molecule is a luciferase, a fluorescent protein (e.g., a GFP, aYFP, etc.), an alkaline phosphatase, or a beta galactosidase. In some embodiments, the luciferase is a firefly luciferase, a Renilla luciferase, or a nanoluciferase. In some embodiments, the promoter and/or element responsive to FcR (e.g., FcγR activation is an NFAT promoter, an AP-1 promoter, an NFκB promoter, a FOXO promoter, a STAT3 promoter, a STAT5 promoter or an IRF promoter. In some embodiments, the element responsive to FcR signaling comprises an NFκB element.

In some embodiments, the reporter cells are phagocytic cells comprising one or more Fc receptors and further comprising a nucleic acid encoding a reporter under the control of a promoter and/or element activated by FcR signaling. In some embodiments, the reporter cells are monocytes comprising one or more Fc receptors and further comprising a nucleic acid encoding a reporter under the control of a promoter and/or element activated by FcR signaling. In some embodiments, the reporter cells are monocytes comprising one or more of FcγRI, FcγRIIa or FcγRIII and further comprising a nucleic acid encoding a reporter under the control of a promoter and/or element activated by FcR signaling. In some embodiments, the reporter cells are monocytes comprising one or more Fc receptors and further comprising a nucleic acid encoding a luciferase reporter under the control of an NF-κB promoter. In some embodiments, the reporter cells are monocytes comprising one or more of FcγRI, FcγRIIa or FcγRIII and further comprising a nucleic acid encoding a luciferase reporter under the control of an NF-κB-promoter. In some embodiments, the reporter cells are THP-1 cells comprising FcγRI, FcγRIIa and/or FcγRIII and further comprising a nucleic acid encoding a luciferase reporter under the control of an NF-κB promoter. In some embodiments, the reporter cells are U-937 cells comprising FcγRI, FcγRIIa and/or FcγRIII and further comprising a nucleic acid encoding a luciferase reporter under the control of an NF-κB promoter.

### Antibody Activity or Potency Assays

In some aspects, the invention provides methods for the activity or potency of polypeptide preparations wherein the polypeptide comprises an antigen binding domain and an Fc receptor binding domain. In some embodiments, the method comprises contacting a preparation of the polypeptide with an immobilized antigen and then contacting the immobilized antigen-polypeptide complex with a population of cells comprising an Fc receptor and nucleic acid encoding a reporter operably linked to a promoter and/or element responsive to Fc receptor activation. Expression of the reporter is indicative of the activity or potency of the polypeptide preparation. In some embodiments, the polypeptide in an antibody or an immunoadhesin. In some embodiments, the reporter is a luciferase, a fluorescent protein, an alkaline phosphatase, a beta lactamase, or a beta galactosidase. In some embodiments, the luciferase is a firefly luciferase, a Renilla luciferase, or a nanoluciferase. In some embodiments, the promoter and/or element responsive to monocyte activation is an NFAT promoter, an AP-1 promoter, or an NFκB promoter. In some embodiments, the promoter and/or element responsive to Fc receptor activation comprises Fc receptor activation responsive elements from any one or more of NFAT, AP-1, and NFκB. In some embodiments, the reporter cells are phagocytic cells. In some embodiments, the reporter cells are monocytes. In some embodiments, the reporter cells are from a cell line. In some embodiments, the cell line is a THP-1 cell line or a U-937 cell line. In some embodiments, the target antigen is beta-amyloid (Aβ) or CD-20. In some embodiments, the Aβ is human Aβ. In some embodiments, the Aβ comprises monomeric and/or oligomeric Aβ. In some embodiments of the invention, the ratio of monomeric to oligomeric Aβ is any of about 10:1, 9:1, 8:1, 7:1, 6:1, 5:1, 4:1, 3:1, 2:1, 1:1, 1:2, 1:3, 1:4, 1:5, 1:6 1:7, 1:8, 1:9; or 1:10. In some embodiments, the human Aβ is Aβ 1-40 or Aβ 1-42. In some embodiments, the polypeptide in crenezumab.

In some embodiments of the invention, the antigen is immobilized on a surface. In some embodiments, the surface is a plate. In some embodiments, the surface is a plate with wells. In some embodiments, the surface is a plate with about any of 96, 182, 288, 384, 480, 576 or 672 wells. In some embodiments, the antigen is immobilized on the surface by adhesion. In some embodiments, the antigen is immobilized on the surface using a streptavidin-biotin system. In some embodiments, streptavidin is linked to the surface and biotin is linked to the antigen and the antigen is subsequently immobilized due to the high affinity of biotin for streptavidin. In some embodiments, the surface is a streptavidin coated plate (e.g., a commercially available streptavidin coated plate). In some embodiments, the surface is a streptavidin coated 96-well plate.

In some embodiments, the antigen in immobilized on a surface at or near the N-terminus of the antigen. In some embodiments, the antigen is immobilized on the surface at or near the C-terminus of the antigen. In some embodiments, the antigen is immobilized on the surface at or near the N-terminus of the antigen and at or near the C-terminus of the antigen such that the antigens are in opposite orientation on the surface. In some embodiments, the antigen is immobilized on the surface at or near the N-terminus of the antigen and at or near the C-terminus of the antigen such that the antigen forms a loop on the surface. In some embodiments, streptavidin is linked to the surface and the antigen comprises biotin at its N-terminus where the biotin binds the streptavidin to immobilize the antigen by its N-terminus. In some embodiments, streptavidin is linked to the surface and the antigen comprises biotin at its C-terminus where the biotin binds the streptavidin to immobilize the antigen by its C-terminus. In some embodiments, streptavidin is linked to the surface and the antigen comprises biotin at its N-terminus and at its C-terminus such that the antigens are in opposite orientation on the surface. In some embodiments, streptavidin is linked to the surface and the antigen comprises biotin at its N-terminus and at its C-terminus where both biotin moieties bind the streptavidin to immobilize the antigen by its N-terminus and by its C-terminus such that the antigen forms a loop on the surface.

In some embodiments, the antigen is conjugated with biotin to form a biotinylated antigen. In some embodiments, the biotinylated antigen is contacted with the streptavidin coated surface wherein the biotinylated antigen is at a concentration of less than about any of 0.1 μg/mL, 0.2 μg/mL, 0.3 μg/mL, 0.4 μg/mL, 0.5 μg/mL, 0.6 μg/mL, 0.7 μg/mL, 0.8 μg/mL, 0.9 μg/mL, 1.0 μg/mL, 1.5 μg/mL, 2.0 μg/mL, 2.5 μg/mL, 3.0 μg/mL, 3.5 μg/mL, 4.0 μg/mL, 4.5 μg/mL, 5.0 μg/mL, 5.5 μg/mL, 6.0 μg/mL, 6.5 μg/mL, 7.0 μg/mL, 7.5 μg/mL, 8.0 μg/mL, 8.5 μg/mL, 9.0 μg/mL, 9.5 μg/mL, 10 μg/mL, 25 μg/mL, or 50 μg/mL. In some embodiments, the biotinylated antigen is contacted with the streptavidin coated multiwell plate wherein about any of the following amounts of biotinylated antigen are added to each well: 10 ng, 20 ng, 30 ng, 40 ng, 50 ng, 60 ng, 70 ng, 80 ng, 90 ng, 0.1 μg 0.2 μg 0.3 μg 0.4 μg 0.5 μg 0.6 μg 0.7 μg 0.8 μg 0.9 μg 1.0 μg or greater than 1.0 μg or any value there between.

In some embodiments, the immobilized antigen is contacted with a composition comprising the polypeptide at a concentration range of any of about 0.01 ng/mL to about 30,000 ng/mL, about 0.01 ng/mL to about 20,000 ng/mL, about 0.01 ng/mL to about 10,000 ng/mL, about 0.05 ng/mL to about 10,000 ng/mL, about 0.1 ng/mL to about 10,000 ng/mL, about 0.5 ng/mL to about 10,000 ng/mL, about 1 ng/mL to about 10,000 ng/mL, about 5 ng/mL to about 10,000 ng/mL, about 10 ng/mL to about 10,000 ng/mL, about 0.01 ng/mL to about 5000 ng/mL, about 0.01 ng/mL to about 4000 ng/mL, about 0.01 ng/mL to about 3000 ng/mL, about 0.01 ng/mL to about 2000 ng/mL, about 0.01 ng/mL to about 1000 ng/mL, about 0.01 ng/mL to about 500 ng/mL, about 0.01 ng/mL to about 100 ng/mL, about 0.01 ng/mL to about 50 ng/mL, about 0.01 ng/mL to about 10 ng/mL, about 0.01 ng/mL to about 5 ng/mL, about 0.1 ng/mL to about 1000 ng/mL, about 0.5 ng/mL to about 1000 ng/mL, about 1 ng/mL to about 100 ng/mL, about 1 ng/mL to about 1000 ng/mL, or about 5 ng/mL to about 5000 ng/mL.

In some embodiments, the immobilized antigen-polypeptide complex is contacted with the reporter cells. In some embodiments, the immobilized antigen-polypeptide complex is contacted with any of about 1×10⁴, 5×10¹, 7.5×10⁴, 1×10⁵, 1.25×10⁵, 1.5×10⁵, 1.75×10⁵, 2×10⁵, 2.25×10⁵, 2.5×10⁵, 2.75×10⁵, 3×10⁵, 3.25×10⁵, 3.5×10⁵, 3.75×10⁵, 4×10⁵, 4.25×10⁵, 4.5×10⁵, 4.75×10⁵, 5×10⁵, 5.5×10⁵, 6×10⁵, 6.5×10⁵, 7×10⁵, 7.5×10⁵, 8×10⁵, 8.5×10⁵, 9×10⁵, 9.5×10⁵, 1×10⁶, 2×10⁶, 3×10⁶, 4×10⁶, or 5×10⁶ reporter cells. In some embodiments, the immobilized antigen-polypeptide complex is contacted with between any of about 1×10⁴ and 5×10⁶, 5×10⁴ and 1×10⁶, 1×10⁵ and 1×10⁶, 1×10⁵ and 2×10⁵, 2×10⁵ and 3×10⁵, 3×10⁵ and 4×10⁵, 4×10⁵ and 5×10⁵, 5×10⁵ and 6×10⁵, 6×10⁵ and 7×10⁵, 7×10⁵ and 8×10⁵, 8×10⁵ and 9×10⁵, or 9×10⁵ and 1×10⁶ reporter cells. In some embodiments, the immobilized antigen-polypeptide complex is contacted with the reporter cells wherein the reporter cells are at a concentration of less than any of about 1×10⁵ cells/ml, 2×10⁵ cells/ml, 3×10⁵ cells/ml, 4×10⁵ cells/ml, 5×10⁵ cells/ml, 6×10⁵ cells/ml, 7×10⁵ cells/ml, 8×10⁵ cells/ml, 9×10⁵ cells/ml, 1×10⁶ cells/ml, 2×10⁶ cells/ml, 2.5×10⁶ cells/ml, 3×10⁶ cells/ml, 4×10⁶ cells/ml, 5×10⁶ cells/ml, 6×10⁶ cells/ml, 7×10⁶ cells/ml, 7.5×10⁶ cells/ml, 8×10⁶ cells/ml, 9×10⁶ cells/ml, or 1×10⁷ cells/ml. In some embodiments, the immobilized antigen-polypeptide complex is contacted with the reporter cells wherein the reporter cells are at a concentration of any of between about 1×10⁵ cells/ml and 1×10⁷ cells/ml, 1×10⁵ cells/ml and 1×10⁶ cells/ml, 5×10⁵ cells/ml and 5×10⁶ cells/ml, or 1×10⁶ cells/ml and 1×10⁷ cells/ml.

In some embodiments, the reporter is detected after more than about any of 1 hr, 2 hr, 3 hr, 4 hr, 5 hr, 6 hr, 7 hr, 8 hr, 9 hr, 10 hr, 12 hr, 16 hr, 20 hr, 24 hr, 24 hr, 28 hr, 30 hr, or 36 hr after contacting the immobilized antigen-polypeptide complex with the reporter cells. In some embodiments, the reporter is detected between any of about 1 hr and about 36 hr, about 1 hr and about 24 hr, about 1 hr and about 12 hr, about 1 hr and about 8 hr, about 1 hr and about 6 hr, about 1 hr and about 4 hr, about 1 hr and about 2 hr, about 4 hr and about 24 hr, about 4 hr and about 12 hr, about 4 hr and about 8 hr, about 8 hr and about 24 hr, about 8 hr and about 12 hr, about 16 hr and about 24 hr, about 16 hr and about 20 hr, or about 20 hr and about 24 hr after contacting the immobilized antigen-polypeptide complex with the reporter cells.

In some aspects the invention provides methods for quantitating the potency of a polypeptide preparation wherein the polypeptide binds a target antigen, the method comprising a) contacting a plurality of populations of immobilized target antigen with different concentrations of the polypeptide preparation to form antigen-polypeptide complexes, b) contacting the antigen-polypeptide complexes with a phagocytic cell, wherein the phagocytic cell comprises an Fcγ receptor and nucleic acid encoding a reporter operably linked to a response element that is responsive to activation by the Fcγ receptor, c) measuring expression of reporter, and d) determining the EC₅₀ of the polypeptide preparation and comparing the EC₅₀ of the polypeptide preparation with the EC₅₀ of a reference standard of the polypeptide of known potency. In some embodiments, the polypeptide is an antibody or an immunoadhesin. In some embodiments, the reporter is a luciferase, a fluorescent protein, an alkaline phosphatase, a beta lactamase, or a beta galactosidase. In some embodiments, the luciferase is a firefly luciferase, a Renilla luciferase, or a nanoluciferase. In some embodiments, the promoter and/or element responsive to Fc receptor activation (e.g., Fcγ receptor activation) wherein the promoter and/or element responsive to Fc receptor activation comprises Fc receptor activation responsive elements for any one or more of NFAT, AP-1, or NFκB. In some embodiments, the reporter cell is a phagocytic cell. In some embodiments, the reporter cells are phagocytic cells. In some embodiments, the reporter cells are monocytes. In some embodiments, the reporter cells are from a cell line. In some embodiments, the cell line is a THP-1 cell line or a U-937 cell line. In some embodiments, the target antigen is beta-amyloid (Aβ) or CD-20. In some embodiments, the Aβ is human Aβ. In some embodiments, the Aβ comprises monomeric and/or oligomeric Aβ. In some embodiments, the human Aβ is Aβ 1-40 or Aβ 1-42. In some embodiments, the polypeptide in crenezumab.

In some embodiments, the EC₅₀ of the polypeptide preparation is compared to the EC₅₀ of a polypeptide preparation of known activity or potency (e.g., a reference standard or reference preparation). As used herein, EC₅₀ refers to the concentration of polypeptide which induces a response halfway between the baseline and maximum after a specified exposure time. In some embodiments, the EC₅₀ of the polypeptide preparation of known activity or potency is determined by generating a standard curve of reporter activity following contact of the immobilized antigen-reference polypeptide complex with the reporter cell. In some embodiments, the standard curve is generated by contacting the population of cells with the reference polypeptide preparation at a plurality of concentrations ranging from about 0.01 ng/mL to about 30,000 ng/mL. In some embodiments, the standard curve is generated by contacting the population of cells with the reference polypeptide preparation at a plurality of concentrations ranging from about 0.01 ng/mL to about 10,000 ng/mL. In some embodiments, the standard curve is generated by contacting the population of cells with the reference polypeptide preparation at a plurality of concentrations ranging from about 0.01 ng/mL to about 15,000 ng/mL. In some embodiments, the standard curve is generated by contacting the population of cells with the reference polypeptide preparation at a plurality of concentrations ranging from about 0.01 ng/mL to about 5,000 ng/mL. In some embodiments, the plurality of concentrations of the reference polypeptide preparation include about any one of 0.01 ng/ml, 0.1 ng/ml, 1 ng/ml, 10 ng/ml, 100 ng/mL, 150 ng/mL, 200 ng/mL, 250 ng/mL, 500 ng/mL, 750 ng/mL, 1 μg/mL, 2.5 μg/mL, 5 μg/mL, 10 μg/mL, 25 μg/mL, 50 μg/mL, 100 μg/mL, 250 μg/mL, or 500 μg/mL. In some embodiments, the plurality of concentrations of the reference polypeptide preparation include about any one of 10 μg/mL, 40 μg/mL, 100 μg/mL, 250 μg/mL, 750 μg/mL, 1000 μg/mL, 1600 μg/mL, 4000 μg/mL, or 10000 μg/mL. In some embodiments, the plurality of concentrations of reference polypeptide preparation is about three, four, five, six, seven, eight, nine, ten, eleven, twelve, thirteen, fourteen, fifteen or more than fifteen concentrations.

In some embodiments, the reporter is detected after more than about any of 1 hr, 2 hr, 3 hr, 4 hr, 5 hr, 6 hr, 7 hr, 8 hr, 9 hr, 10 hr, 12 hr, 16 hr, 20 hr, 24 hr, 26 hr, 28 hr, 30 hr, or 36 hr after contacting the cells with the composition. In some embodiments, the reporter is detected between any of about 1 hr and about 24 hr, about 1 hr and about 12 hr, about 1 hr and about 8 hr, about 1 hr and about 6 hr, about 1 hr and about 4 hr, about 1 hr and about 2 hr, about 4 hr and about 24 hr, about 4 hr and about 12 hr, about 4 hr and about 8 hr, about 8 hr and about 24 hr, about 8 hr and about 12 hr, about 16 hr and about 24 hr, about 16 hr and about 20 hr, or about 20 hr and about 24 hr after contacting the cells with the composition.

In some embodiments of the invention, the methods further comprising calculating the potency based on the EC₅₀ of the polypeptide preparation using a multi-parameter logistic fit against the reference standard. In some embodiments, the multi-parameter logistic fit is a 3-parameter, 4-parameter, or 5-parameter logistic fit. Such methods of multi-parameter fit our known in the art.

In some embodiments, the potency of the polypeptide preparation is based on the EC₅₀ of the polypeptide preparation using a 4-parameter logisitic fit as follows:

Using the luminescence value measured in relative light units (RLU) of each individual well, the average well value for each standard (ST) and test article (control and sample(s); TA) concentration is calculated, wherein replicate wells are tested.

A dose response curve for standard, control and samples is generated by plotting the average well value for each concentration on the y-axis (linear scale) versus the concentration on the x-axis (logarithmic scale).

A 4-parameter logistic curve-fitting program is used to generate separate curves for ST and each TA. The 4-parameter logistic curve-fitting equation is:

$$y = D + \frac{A - D}{1 + {( \frac{x}{C} )}^{B}}$$

### Where:

x=concentration of ST or TA
y=average well value response (RLU)
A=Zero dose response (lower asymptote=LA):
B=slope
C=EC₅₀ (half-maximal effective concentration)
D=Maximum dose response (upper asymptote=UA)
Calculate the coefficient of determination (RV) for each curve.

Calculate the fold response of the standard, product control and sample curves.

Fold Response=UA÷LA

The slope ratio is calculated as follows:

$$Slope ratio = \vert \frac{( {D}_{TA} - {A}_{TA} ) \times {B}_{TA}}{( {D}_{ST} - {A}_{ST} ) \times {B}_{ST}} \vert$$

The upper asymptote percent difference is calculated as follows

$$U A D = {100}^{*} \vert \frac{{D}_{T A} - {D}_{S T}}{{D}_{S T} - {A}_{S T}} \vert$$

The lower asymptote percent difference is calculated as follows

$$L A D = {100}^{*} \vert \frac{{A}_{T A} - {A}_{S T}}{{D}_{S T} - {A}_{S T}} \vert$$

The relative potency of a test article is calculated using a 4-parameter parallel curve analysis. Generate a constrained 4-P parallel curve for ST and each TA with a common set of parameters: slope (parameter B), upper asymptote (parameter D) and lower asymptote (parameter A). The resulting curve equations for standard (ST) and test article (TA) are:

$${y}_{S T} = D + \frac{A - D}{1 + {( \frac{x}{{C}_{S T}} )}^{B}} \qquad {y}_{T A} = D + \frac{A - D}{1 + {( \frac{ρ x}{{C}_{S T}} )}^{B}}$$

Where:
x=concentration of antibody
yST=standard RLU
yTA=test article RLU
A=common lower asymptote
B=common slope
CST=standard EC₅₀
D=common upper asymptote
ρ=sample relative potency (the relative potency is the ratio of EC₅₀ of ST over EC₅₀ of TA)

Calculate the potency of the test article according to the equation:

Potency=ρ*Activity of the reference standard

### Kits

In some aspects of the invention, a kit or article of manufacture is provided for use in assays to determine the activity or potency of a polypeptide preparation, comprising a container which holds a composition comprising engineered cells comprising nucleic acid encoding a reporter operably linked to a promoter and/or elements that are responsive to Fc receptor activation as described herein, and optionally provides instructions for its use. In some embodiments, the kit further comprises a container which holds a reference polypeptide preparation assay standard (a polypeptide preparation of known activity or potency), and/or a container which holds a polypeptide preparation reference standard. In some embodiments, the kit further comprises a container or surface which comprises an immobilized antigen. In some embodiments, the reporter is a luciferase, a fluorescent protein, an alkaline phosphatase, a beta lactamase, or a beta galactosidase. In some embodiments, the luciferase is a firefly luciferase, a Renilla luciferase, or a nanoluciferase. In some embodiments, the promoter and/or element responsive to Fc receptor activation comprises an Fc receptor activation responsive elements from any one or more of NFAT, AP-1, NFκB, FOXO, STAT3, STAT5 and IRF. In some embodiments, the reporter cell is a phagocytic cell. In some embodiments, the phagocytic cell is a monocyte. In some embodiments, the phagocytic cell is from a cell line. In some embodiments, the phagocytic cell line is a THP-1 cell line or a U-937 cell line.

The containers hold the formulations and the labels on, or associated with, the containers may indicate directions for use. The article of manufacture may further include other materials desirable from a commercial and user standpoint, including other buffers, diluents, cultureware, reagents for detecting reporter molecules, and package inserts with instructions for use.

In some aspects of the invention, a kit or article of manufacture is provided comprising a container which holds a composition comprising an antigen conjugated with biotin, and optionally provides instructions for its use. In some embodiments, the kit further provides a reference polypeptide assay standard (a polypeptide preparation of known activity or potency), and/or an antigen-binding control. The containers hold the formulations and the labels on, or associated with, the containers may indicate directions for use. The article of manufacture may further include other materials desirable from a commercial and user standpoint, including other buffers, diluents, cultureware, reagents for detecting reporter molecules, and package inserts with instructions for use.

### Polypeptides

The polypeptides to be analyzed using the methods described herein are generally produced using recombinant techniques. Methods for producing recombinant proteins are described, e.g., in U.S. Pat. Nos. 5,534,615 and 4,816,567, specifically incorporated herein by reference. In some embodiments, the protein of interest is produced in a CHO cell (see. e.g. WO 94/11026). In some embodiments, the polypeptide of interest is produced in an E. coli cell. See, e.g., U.S. Pat. Nos. 5,648,237; 5,789,199, and 5,840,523, which describes translation initiation region (T1R) and signal sequences for optimizing expression and secretion. See also Charlton, Methods in Molecular Biology, Vol. 248 (B. K. C. Lo, ed., Humana Press, Totowa, N.J., 2003), pp. 245-254, describing expression of polypeptide fragments in E. coli. When using recombinant techniques, the polypeptides can be produced intracellularly, in the periplasmic space, or directly secreted into the medium.

The polypeptides may be recovered from culture medium or from host cell lysates. Cells employed in expression of the polypeptides can be disrupted by various physical or chemical means, such as freeze-thaw cycling, sonication, mechanical disruption, or cell lysing agents. If the polypeptide is produced intracellularly, as a first step, the particulate debris, either host cells or lysed fragments, are removed, for example, by centrifugation or ultrafiltration. Carter et al., Bio/Technology 10: 163-167 (1992) describe a procedure for isolating polypeptides which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the polypeptide is secreted into the medium, supernatants from such expression systems are generally first concentrated using a commercially available polypeptide concentration filter, for example, an Amicon® or Millipore Pellicon® ultrafiltration unit. A protease inhibitor such as PMSF may be included in any of the foregoing steps to inhibit proteolysis and antibiotics may be included to prevent the growth of adventitious contaminants.

In some embodiments, the polypeptide in the composition comprising the polypeptide and one or more contaminants has been purified or partially purified prior to analysis by the methods of the invention. For example, the polypeptide of the methods is in an eluent from an affinity chromatography, a cation exchange chromatography, an anion exchange chromatography, a mixed mode chromatography and a hydrophobic interaction chromatography. In some embodiments, the polypeptide is in an eluent from a Protein A chromatography.

Examples of polypeptides that may be analyzed by the methods of the invention include but are not limited to immunoglobulins, immunoadhesins, antibodies, enzymes, hormones, fusion proteins, Fe-containing proteins, immunoconjugates, cytokines and interleukins.

### (A) Antibodies

In some embodiments of any of the methods described herein, the polypeptide for use in any of the methods of analyzing polypeptides and formulations comprising the polypeptides by the methods described herein is an antibody or immunoadhesin. In some embodiments, the antigen target of the polypeptide of the invention is A-beta or CD20.

Other exemplary antibodies include those selected from, and without limitation, anti-estrogen receptor antibody, anti-progesterone receptor antibody, anti-p53 antibody, anti-HER-2/neu antibody, anti-EGFR antibody, anti-cathepsin D antibody, anti-Bcl-2 antibody, anti-E-cadherin antibody, anti-CA125 antibody, anti-CA15-3 antibody, anti-CA19-9 antibody, anti-c-erbB-2 antibody, anti-P-glycoprotein antibody, anti-CEA antibody, anti-retinoblastoma protein antibody, anti-ras oncoprotein antibody, anti-Lewis X antibody, anti-Ki-67 antibody, anti-PCNA antibody, anti-CD3 antibody, anti-CD4 antibody; anti-CD5 antibody, anti-CD7 antibody, anti-CD8 antibody, anti-CD9/p24 antibody, anti-CD10 antibody, anti-CD11a antibody, anti-CD11e antibody, anti-CD13 antibody, anti-CD14 antibody, anti-CD15 antibody, anti-CD19 antibody, anti-CD22 antibody, anti-CD23 antibody, anti-CD30 antibody, anti-CD31 antibody, anti-CD33 antibody, anti-CD34 antibody, anti-CD35 antibody, anti-CD38 antibody, anti-CD41 antibody, anti-LCA/CD45 antibody, anti-CD45RO antibody, anti-CD45RA antibody, anti-CD39 antibody, anti-CD100 antibody, anti-CD95/Fas antibody, anti-CD99 antibody, anti-CD106 antibody, anti-ubiquitin antibody, anti-CD71 antibody, anti-c-myc antibody, anti-cytokcratins antibody, anti-vimentin antibody, anti-HPV proteins antibody, anti-kappa light chains antibody, anti-lambda light chains antibody, anti-melanosomes antibody, anti-prostate specific antigen antibody, anti-S-100 antibody, anti-tau antigen antibody, anti-fibrin antibody, anti-keratins antibody, anti-TebB2 antibody, anti-STEAP antibody, and anti-Tn-antigen antibody.

### (i) Monoclonal Antibodies

In some embodiments, the antibodies are monoclonal antibodies. Monoclonal antibodies are obtained from a population of substantially homogeneous antibodies, i.e., the individual antibodies comprising the population are identical and/or bind the same epitope except for possible variants that arise during production of the monoclonal antibody, such variants generally being present in minor amounts. Thus, the modifier “monoclonal” indicates the character of the antibody as not being a mixture of discrete or polyclonal antibodies.

For example, the monoclonal antibodies may be made using the hybridoma method first described by Kohler et al., Nature 256:495 (1975), or may be made by recombinant DNA methods (U.S. Pat. No. 4,816,567).

In the hybridoma method, a mouse or other appropriate host animal, such as a hamster, is immunized as herein described to elicit lymphocytes that produce or are capable of producing antibodies that will specifically bind to the polypeptide used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

In some embodiments, the myeloma cells are those that fuse efficiently, support stable high-level production of antibody by the selected antibody-producing cells, and are sensitive to a medium such as HAT medium. Among these, in some embodiments, the myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol. 133:3001 (1984); Brodeur et al., Monoclonal Antibody Production Techniques and Applications pp. 51-63 (Marcel Dekker, Inc.; New York, 1987)).

Culture medium in which hybridoma cells are growing is assayed for production of monoclonal antibodies directed against the antigen. In some embodiments, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al., Anal. Biochem. 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, polypeptide A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). In some embodiments, the hybridoma cells serve as a source of such DNA. Once isolated, the DNA may be placed into expression vectors, which are then transfected into host cells such as E. coli cells, simian COS cells; Chinese Hamster Ovary (CHO) cells, or myeloma cells that do not otherwise produce immunoglobulin polypeptide, to obtain the synthesis of monoclonal antibodies in the recombinant host cells. Review articles on recombinant expression in bacteria of DNA encoding the antibody include Skerra et al., Curr. Opinion in Immunol. 5:256-262 (1993) and Plückthun, Immunol. Revs., 130:151-188 (1992).

In a further embodiment, antibodies or antibody fragments can be isolated from antibody phage libraries generated using the techniques described in McCafferty et al., Nature 348:552-554 (1990). Clackson et al., Nature 352:624-628 (1991) and Marks et al., J. Mol. Biol. 222:581-597 (1991) describe the isolation of murine and human antibodies, respectively, using phage libraries. Subsequent publications describe the production of high affinity (nM range) human antibodies by chain shuffling (Marks et al., Bio/Technology 10:779-783 (1992)), as well as combinatorial infection and in vivo recombination as a strategy for constructing very large phage libraries (Waterhouse et al., Nuc. Acids. Res. 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light chain constant domains in place of the homologous murine sequences (U.S. Pat. No. 4,816,567; Morrison et al., Proc. Natl Acad. Sci. USA 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

In some embodiments of any of the methods described herein, the antibody is IgA, IgD, IgE, IgG, or IgM. In some embodiments, the antibody is an IgG monoclonal antibody.

### (ii) Humanized Antibodies

In some embodiments, the antibody is a humanized antibody. Methods for humanizing non-human antibodies have been described in the art. In some embodiments, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence that is closest to that of the rodent is then accepted as the human framework region (FR) for the humanized antibody (Sims et al., J. Immunol. 151:2296 (1993); Chothia et al., J. Mol. Biol. 196:901 (1987)). Another method uses a particular framework region derived from the consensus sequence of all human antibodies of a particular subgroup of light or heavy chain variable regions. The same framework may be used for several different humanized antibodies (Carter et al., Proc. Natl. Acad. Sci. USA 89:4285 (1992); Presta et al., J. Immunol. 151:2623 (1993)).

It is further important that antibodies be humanized with retention of high affinity for the antigen and other favorable biological properties. To achieve this goal, in some embodiments of the methods, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

(iii) Human Antibodies

In some embodiments, the antibody is a human antibody. As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA 90:2551 (1993); Jakobovits et al., Nature 362:255-258 (1993); Bruggermann et al., Year in Immuno. 7:33 (1993); and U.S. Pat. Nos. 5,591,669; 5,589,369; and 5,545,807.

Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat polypeptide gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S. and Chiswell, David J., Current Opinion in Structural Biology 3:564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See also, U.S. Pat. Nos. 5,565,332 and 5,573,905.

Human antibodies may also be generated by in vitro activated B cells (see U.S. Pat. Nos. 5,567,610 and 5,229,275).

### (iv) Antibody Fragments

In some embodiments, the antibody is an antibody fragment. In some embodiments, the antibody is an antibody fragment comprising an Fc receptor binding domain. Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science 229:81 (1985)). However, these fragments can now be produced directly by recombinant host cells. For example, the antibody fragments can be isolated from the antibody phage libraries discussed above.

In some embodiments, fragments of the antibodies described herein are provided. In some embodiments, the antibody fragment is an antigen binding fragment. In some embodiments, the antibody fragment is an antigen binding fragment comprising an Fc receptor binding domain. In some embodiments, the antibody fragment is an antigen binding fragment comprising an Fcγ receptor binding domain.

### (v) Bispecific Antibodies

In some embodiments, the antibody is a bispecific antibody. Bispecific antibodies are antibodies that have binding specificities for at least two different epitopes. Exemplary bispecific antibodies may bind to two different epitopes. Alternatively, a bispecific antibody binding arm may be combined with an arm that binds to a triggering molecule on a leukocyte such as a T-cell receptor molecule (e.g. CD2 or CD3), or Fc receptors for IgG (FcγR), such as FcγRI (CD64), FcγRII (CD32) and FcγRIII (CD16) so as to focus cellular defense mechanisms to the cell. Bispecific antibodies can be prepared as full length antibodies or antibody fragments (e.g. F(ab′)2 bispecific antibodies).

Methods for making bispecific antibodies are known in the art. Traditional production of full length bispecific antibodies is based on the coexpression of two immunoglobulin heavy chain-light chain pairs, where the two chains have different specificities (Millstein et al., Nature 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. In some embodiments, the fusion is with an immunoglobulin heavy chain constant domain, comprising at least part of the hinge, CH2, and CH3 regions. In some embodiments, the first heavy chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios arc of no particular significance.

In some embodiments of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology 121:210 (1986).

According to another approach described in U.S. Pat. No. 5,731,168, the interface between a pair of antibody molecules can be engineered to maximize the percentage of heterodimers that are recovered from recombinant cell culture. In some embodiments, the interface comprises at least a part of the C_{H}3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 0308936). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science 229: 81 (1985) describe a procedure wherein intact antibodies are proteolytically cleaved to generate F(ab′)2 fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy chain variable domain (V_{H}) connected to a light chain variable domain (V_{L}) by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol. 152:5368 (1994).

Antibodies with more than two valencies are contemplated. For example, trispecific antibodies can be prepared. Tutt et al., J. Immunol. 147: 60 (1991).

### (v) Multivalent Antibodies

In some embodiments, the antibodies are multivalent antibodies. A multivalent antibody may be internalized (and/or catabolized) faster than a bivalent antibody by a cell expressing an antigen to which the antibodies bind. The antibodies provided herein can be multivalent antibodies (which are other than of the IgM class) with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1)n-VD2-(X2) n-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

In some embodiments, the antibody is a multispecific antibody. Example of multispecific antibodies include, but are not limited to, an antibody comprising a heavy chain variable domain (V_{H}) and a light chain variable domain (V_{L}), where the V_{H}V_{L} unit has polyepitopic specificity, antibodies having two or more V_{L} and V_{H} domains with each V_{H}V_{L} unit binding to a different epitope, antibodies having two or more single variable domains with each single variable domain binding to a different epitope, full length antibodies, antibody fragments such as Fab, Fv, dsFv, scFv, diabodies, bispecific diabodies, triabodies, tri-functional antibodies, antibody fragments that have been linked covalently or non-covalently. In some embodiment that antibody has polyepitopic specificity; for example, the ability to specifically bind to two or more different epitopes on the same or different target(s). In some embodiments, the antibodies are monospecific; for example, an antibody that binds only one epitope. According to one embodiment the multispecific antibody is an IgG antibody that binds to each epitope with an affinity of 5 μM to 0.001 pM, 3 μM to 0.001 pM, 1 μM to 0.001 pM, 0.5 μM to 0.001 pM, or 0.1 μM to 0.001 pM.

### (vi) Other Antibody Modifications

It may be desirable to modify the antibody provided herein with respect to effector function, e.g., so as to enhance antigen-dependent cell-mediated cytotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J., Immunol. 148:2918-2922 (1992). Homodimeric antibodies with enhanced anti-tumor activity may also be prepared using heterobifunctional cross-linkers as described in Wolff et al., Cancer Research 53:2560-2565 (1993). Alternatively, an antibody can be engineered which has dual Fc regions and may thereby have enhanced complement mediated lysis and ADCC capabilities. See Stevenson et al., Anti-Cancer Drug Design 3:219-230 (1989).

For increasing serum half-life of the antibody, amino acid alterations can be made in the antibody as described in US 2006/0067930, which is hereby incorporated by reference in its entirety.

### (B) Polypeptide Variants and Modifications

Amino acid sequence modification(s) of the polypeptides, including antibodies, described herein may be used in the methods of purifying polypeptides (e.g., antibodies) described herein.

### (i) Variant Polypeptides

“Polypeptide variant” means a polypeptide, preferably an active polypeptide, as defined herein having at least about 80% amino acid sequence identity with a full-length native sequence of the polypeptide, a polypeptide sequence lacking the signal peptide, an extracellular domain of a polypeptide, with or without the signal peptide. Such polypeptide variants include, for instance, polypeptides wherein one or more amino acid residues are added, or deleted, at the N or C-terminus of the full-length native amino acid sequence. Ordinarily, a TAT polypeptide variant will have at least about 80% amino acid sequence identity, alternatively at least about any of 85%, 90%, 95%, 96%, 97%, 98%, or 99% amino acid sequence identity, to a full-length native sequence polypeptide sequence, a polypeptide sequence lacking the signal peptide, an extracellular domain of a polypeptide, with or without the signal peptide. Optionally, variant polypeptides will have no more than one conservative amino acid substitution as compared to the native polypeptide sequence, alternatively no more than about any of 2, 3, 4, 5, 6, 7, 8, 9, or 10 conservative amino acid substitutions as compared to the native polypeptide sequence.

The variant polypeptide may be truncated at the N-terminus or C-terminus, or may lack internal residues, for example, when compared with a full length native polypeptide. Certain variant polypeptides may lack amino acid residues that are not essential for a desired biological activity. These variant polypeptides with truncations, deletions, and insertions may be prepared by any of a number of conventional techniques. Desired variant polypeptides may be chemically synthesized. Another suitable technique involves isolating and amplifying a nucleic acid fragment encoding a desired variant polypeptide, by polymerase chain reaction (PCR). Oligonucleotides that define the desired termini of the nucleic acid fragment are employed at the 5′ and 3′ primers in the PCR. Preferably, variant polypeptides share at least one biological and/or immunological activity with the native polypeptide disclosed herein.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody to an enzyme or a polypeptide which increases the serum half-life of the antibody.

For example, it may be desirable to improve the binding affinity and/or other biological properties of the polypeptide. Amino acid sequence variants of the polypeptide are prepared by introducing appropriate nucleotide changes into the antibody nucleic acid, or by peptide synthesis. Such modifications include, for example, deletions from, and/or insertions into and/or substitutions of, residues within the amino acid sequences of the polypeptide. Any combination of deletion, insertion, and substitution is made to arrive at the final construct, provided that the final construct possesses the desired characteristics. The amino acid changes also may alter post-translational processes of the polypeptide (e.g., antibody), such as changing the number or position of glycosylation sites.

Guidance in determining which amino acid residue may be inserted, substituted or deleted without adversely affecting the desired activity may be found by comparing the sequence of the polypeptide with that of homologous known polypeptide molecules and minimizing the number of amino acid sequence changes made in regions of high homology.

A useful method for identification of certain residues or regions of the polypeptide (e.g., antibody) that are preferred locations for mutagenesis is called “alanine scanning mutagenesis” as described by Cunningham and Wells, Science 244:1081-1085 (1989). Here, a residue or group of target residues are identified (e.g., charged residues such as Arg, Asp, His, Lys, and Glu) and replaced by a neutral or negatively charged amino acid (most preferably Alanine or Polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antibody variants are screened for the desired activity.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in the Table 1 below under the heading of “exemplary substitutions.” If such substitutions result in a change in biological activity, then more substantial changes, denominated “substitutions” in the Table 1, or as further described below in reference to amino acid classes, may be introduced and the products screened.

TABLE 1

| Original | | Exemplary |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu, Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Substantial modifications in the biological properties of the polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain Amino acids may be grouped according to similarities in the properties of their side chains (in A. L. Lehninger, Biochemistry second ed., pp. 73-75, Worth Publishers, New York (1975)):

(1) non-polar: Ala (A), Val (V), Leu (L), Ile (I), Pro (P), Phe (F), Trp (W), Met (M)
(2) uncharged polar: Gly (G), Ser (S), Thr (T), Cys (C), Tyr (Y), Asn (N), Gln (Q)
(3) acidic: Asp (D), Glu (E)
(4) basic: Lys (K), Arg (R), His (H)

Alternatively, naturally occurring residues may be divided into groups based on common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the polypeptide to improve its stability (particularly where the antibody is an antibody fragment such as an Fv fragment).

A particularly preferred type of substitutional variant involves substituting one or more hypervariable region residues of a parent antibody (e.g., a humanized antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and target. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the polypeptide alters the original glycosylation pattern of the antibody. The polypeptide may comprise non-amino acid moieties. For example, the polypeptide may be glycosylated. Such glycosylation may occur naturally during expression of the polypeptide in the host cell or host organism, or may be a deliberate modification arising from human intervention. By altering is meant deleting one or more carbohydrate moieties found in the polypeptide, and/or adding one or more glycosylation sites that are not present in the polypeptide.

Glycosylation of polypeptide is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the polypeptide is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Removal of carbohydrate moieties present on the polypeptide may be accomplished chemically or enzymatically or by mutational substitution of codons encoding for amino acid residues that serve as targets for glycosylation. Enzymatic cleavage of carbohydrate moieties on polypeptides can be achieved by the use of a variety of endo- and exo-glycosidases.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains, acetylation of the N-terminal amine, and amidation of any C-terminal carboxyl group.

### (ii) Chimeric Polypeptides

The polypeptide described herein may be modified in a way to form chimeric molecules comprising the polypeptide fused to another, heterologous polypeptide or amino acid sequence. In some embodiments, a chimeric molecule comprises a fusion of the polypeptide with a tag polypeptide which provides an epitope to which an anti-tag antibody can selectively bind. The epitope tag is generally placed at the amino- or carboxyl-terminus of the polypeptide. The presence of such epitope-tagged forms of the polypeptide can be detected using an antibody against the tag polypeptide. Also, provision of the epitope tag enables the polypeptide to be readily purified by affinity purification using an anti-tag antibody or another type of affinity matrix that binds to the epitope tag.

In an alternative embodiment, the chimeric molecule may comprise a fusion of the polypeptide with an immunoglobulin or a particular region of an immunoglobulin. A bivalent form of the chimeric molecule is referred to as an “immunoadhesin.”

As used herein, the term “immunoadhesin” designates antibody-like molecules which combine the binding specificity of a heterologous polypeptide with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG1, IgG2, IgG3, or IgG4 subtypes, IgA (including IgA1 and IgA2), IgE, IgD or IgM.

The Ig fusions preferably include the substitution of a soluble (transmembrane domain deleted or inactivated) form of a polypeptide in place of at least one variable region within an Ig molecule. In a particularly preferred embodiment, the immunoglobulin fusion includes the hinge, CH2 and CH3, or the hinge, CH₁, CH₂ and CH₃ regions of an IgG1 molecule.

(iii) Polypeptide Conjugates

The polypeptide for use in polypeptide formulations may be conjugated to a cytotoxic agent such as a chemotherapeutic agent, a growth inhibitory agent, a toxin (e.g., an enzymatically active toxin of bacterial, fungal, plant, or animal origin, or fragments thereof), or a radioactive isotope (i.e., a radioconjugate).

Chemotherapeutic agents useful in the generation of such conjugates can be used. In addition, enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated polypeptides. Examples include ²¹²Bi, ¹³¹I, ¹³¹In, ⁹⁰Y, and ¹⁸⁶Re. Conjugates of the polypeptide and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucicotide to the polypeptide.

Conjugates of a polypeptide and one or more small molecule toxins, such as a calicheamicin, maytansinoids, a trichothene, and CC1065, and the derivatives of these toxins that have toxin activity, are also contemplated herein. Maytansinoids are mitototic inhibitors which act by inhibiting tubulin polymerization.

Maytansine was first isolated from the east African shrub Maytenus serrata. Subsequently, it was discovered that certain microbes also produce maytansinoids, such as maytansinol and C-3 maytansinol esters. Synthetic maytansinol and derivatives and analogues thereof are also contemplated. There are many linking groups known in the art for making polypeptide-maytansinoid conjugates, including, for example, those disclosed in U.S. Pat. No. 5,208,020. The linking groups include disufide groups, thioether groups, acid labile groups, photolabile groups, peptidase labile groups, or esterase labile groups, as disclosed in the above-identified patents; disulfide and thioether groups being preferred.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hyrdoxymethyl, the C-15 position modified with a hydroxyl group, and the C-20 position having a hydroxyl group. In a preferred embodiment, the linkage is formed at the C-3 position of maytansinol or a maytansinol analogue.

Another conjugate of interest comprises a polypeptide conjugated to one or more calicheamicin molecules. The calicheamicin family of antibiotics is capable of producing double-stranded DNA breaks at sub-picomolar concentrations. For the preparation of conjugates of the calicheamicin family, see, e.g., U.S. Pat. No. 5,712,374. Structural analogues of calicheamicin which may be used include, but are not limited to, γ₁^{I}, α₂^{I}, α₃^{I}, N-acetyl-γ₁^{I}, PSAG and θ₁^{I}. Another anti-tumor drug that the antibody can be conjugated is QFA which is an antifolate. Both calicheamicin and QFA have intracellular sites of action and do not readily cross the plasma membrane. Therefore, cellular uptake of these agents through polypeptide (e.g., antibody) mediated internalization greatly enhances their cytotoxic effects.

Other antitumor agents that can be conjugated to the polypeptides described herein include BCNU, streptozoicin, vincristine and 5-fluorouracil, the family of agents known collectively LL-E33288 complex, as well as esperamicins.

In some embodiments, the polypeptide may be a conjugate between a polypeptide and a compound with nucleolytic activity (e.g., a ribonuclease or a DNA endonuclease such as a deoxyribonuclease; DNase).

In yet another embodiment, the polypeptide (e.g., antibody) may be conjugated to a “receptor” (such streptavidin) for utilization in tumor pre-targeting wherein the polypeptide receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

In some embodiments, the polypeptide may be conjugated to a prodrug-activating enzyme which converts a prodrug (e.g., a peptidyl chemotherapeutic agent) to an active anti-cancer drug. The enzyme component of the immunoconjugate includes any enzyme capable of acting on a prodrug in such a way so as to convert it into its more active, cytotoxic form.

Enzymes that are useful include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosinc into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as β-galactosidase and neuraminidase useful for converting glycosylated prodrugs into free drugs; β-lactamase useful for converting drugs derivatized with β-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert the prodrugs into free active drugs.

### (iv) Other

Another type of covalent modification of the polypeptide comprises linking the polypeptide to one of a variety of nonproteinaceous polymers, e.g., polyethylene glycol, polypropylene glycol, polyoxyalkylenes, or copolymers of polyethylene glycol and polypropylene glycol. The polypeptide also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example; liposomes, albumin microspheres, microemulsions, nano-particles and nanocapsules), or in macroemulsions. Such techniques are disclosed in Remington's Pharmaceutical Sciences, 18th edition, Gennaro, A. R., Ed., (1990).

### Obtaining Polypeptides for Use in the Formulations and Methods

The polypeptides used in the methods of analysis described herein may be obtained using methods well-known in the art, including the recombination methods. The following sections provide guidance regarding these methods.

### (A) Polynucleotides

“Polynucleotide,” or “nucleic acid,” as used interchangeably herein, refer to polymers of nucleotides of any length, and include DNA and RNA.

Polynucleotides encoding polypeptides may be obtained from any source including, but not limited to, a cDNA library prepared from tissue believed to possess the polypeptide mRNA and to express it at a detectable level. Accordingly, polynucleotides encoding polypeptide can be conveniently obtained from a cDNA library prepared from human tissue. The polypeptide-encoding gene may also be obtained from a genomic library or by known synthetic procedures (e.g., automated nucleic acid synthesis).

For example, the polynucleotide may encode an entire immunoglobulin molecule chain, such as a light chain or a heavy chain. A complete heavy chain includes not only a heavy chain variable region (VH) but also a heavy chain constant region (CH), which typically will comprise three constant domains: C_{H}1, C_{H}2 and C_{H}3; and a “hinge” region. In some situations, the presence of a constant region is desirable. In some embodiments, the polynucleotide encodes one or more immunoglobulin molecule chains of a TDB.

Other polypeptides which may be encoded by the polynucleotide include antigen-binding antibody fragments such as single domain antibodies (“dAbs”), Fv, scFv, Fab′ and F(ab′)2 and “minibodies.” Minibodies are (typically) bivalent antibody fragments from which the C_{H}1 and C_{K} or C_{L} domain has been excised. As minibodies are smaller than conventional antibodies they should achieve better tissue penetration in clinical/diagnostic use, but being bivalent they should retain higher binding affinity than monovalent antibody fragments, such as dAbs. Accordingly, unless the context dictates otherwise, the term “antibody” as used herein encompasses not only whole antibody molecules but also antigen-binding antibody fragments of the type discussed above. Preferably each framework region present in the encoded polypeptide will comprise at least one amino acid substitution relative to the corresponding human acceptor framework. Thus, for example, the framework regions may comprise, in total, three, four, five, six, seven, eight, nine, ten, eleven, twelve, thirteen, fourteen, or fifteen amino acid substitutions relative to the acceptor framework regions.

### EXEMPLARY EMBODIMENTS

1. A method for determining the activity of a polypeptide wherein the polypeptide binds a target antigen and the polypeptide comprises an Fc receptor binding domain, the method comprising

a) contacting an immobilized target antigen with the polypeptide preparation to form an antigen-polypeptide complex,

b) contacting the antigen-polypeptide complex with a phagocytic cell, wherein the phagocytic cell comprises an Fcγ receptor and nucleic acid encoding a reporter operably linked to a response element that is responsive to activation by the Fcγ receptor;

wherein expression of the reporter indicates activity of the polypeptide.

2. A method for quantitating the potency of a polypeptide preparation wherein the polypeptide binds a target antigen, the method comprising

a) contacting a plurality of populations of immobilized target antigen with different concentrations of the polypeptide preparation to form antigen-polypeptide complexes,

b) contacting the antigen-polypeptide complexes with a phagocytic cell, wherein the phagocytic cell comprises an Fcγ receptor and nucleic acid encoding a reporter operably linked to a response element that is responsive to activation by the Fcγ receptor,

c) measuring expression of reporter, and

d) determining the EC₅₀ of the polypeptide preparation and comparing the EC₅₀ of the polypeptide preparation with the EC₅₀ of a reference standard of the polypeptide of known potency.

3. The method of embodiment 2, further comprising calculating the potency based on the EC₅₀ of the polypeptide preparation using a multi-parameter logistic fit against the reference standard.

4. The method of embodiment 3, wherein the multi-parameter logistic fit is a 3-parameter, 4-parameter, or 5-parameter logistic fit.

5. The method of any one of embodiments 2-4, wherein the EC50 of the reference standard is determined at the same time as the EC₅₀ of the polypeptide preparation.

6. The method of any one of embodiments 1-5, wherein the reporter is a luciferase or a fluorescent protein.

7. The method of embodiment 6, wherein the luciferase is a firefly luciferase, a Renilla luciferase, or a nanoluciferase.

8. The method of any one of embodiments 1-7, wherein the response element that is responsive to activation by the Fcγ receptor is an NFκB response element, an NEAT response element, an AP-1 response element, or an ERK-responsive transcription factor (e.g. Elk1).

9. The method of any one of embodiments 1-8, wherein the phagocytic cell is a monocyte.

10. The method of any one of embodiments 1-9, wherein the phagocytic cell is from a cell line.

11. The method of embodiment 10, wherein the cell line is a THP-1 cell line or a U-937 cell line.

12. The method of any one of embodiments 1-11, wherein the Fcγ receptor is a FcγRI (CD64) or FcγRIIa (CD32a) or FcγRIII (CD16).

13. The method of any one of embodiments 1-12, wherein the phagocytic cell is engineered to overexpress a Fcγ receptor.

14. The method of embodiment 13, wherein the phagocytic cell is engineered to overexpress a FcγRIIa.

15. The method of any one of embodiments 1-14, wherein the phagocytic cell does not express FcγRIII.

16. The method of any one of embodiments 1-15, wherein the target antigen is beta-amyloid (Aβ) or CD20.

17. The method of embodiment 16, wherein the target antigen is beta-amyloid (Aβ).

18. The method of embodiment 17, wherein the Aβ is human Aβ.

19. The method of embodiment 17 or 18, wherein the Aβ comprises monomeric and/or oligomeric A3.

20. The method of embodiment 17, wherein the human Aβ is Aβ 1-40 or Aβ 1-42.

21. The method of any one of embodiments 1-20, wherein the polypeptide comprises a full length Fc domain or an FcR-binding fragment of an Fc domain.

22. The method of any one of embodiments 1-21, wherein the polypeptide specifically binds Aβ.

23. The method of any one of embodiments 1-22, wherein the polypeptide is an antibody or an immunoadhesin.

24. The method of embodiment 22 or 23, wherein the polypeptide in crenezumab.

25. The method of any one of embodiments 1-24, wherein the target antigen is immobilized on a surface.

26. The method of embodiment 25, wherein the surface is a plate.

27. The method of embodiment 26, wherein the plate is a multi-well plate.

28. The method of any one of embodiments 25-27, wherein the antigen is immobilized to the surface at or near its N-terminus, at or near its C-terminus, or at or near its N-terminus and at or near its C-terminus.

29. The method of any one of embodiments 25-28, wherein the target antigen is immobilized on the surface using a biotin-streptavidin system.

30. The method of embodiment 29, wherein the target antigen is bound to biotin and the surface comprises bound streptavidin.

31. The method of embodiment 29 or 30, wherein the target antigen is bound to biotin at or near its N-terminus, at or near its C-terminus, or at or near its N-terminus and its C-terminus.

32. The method of any one of embodiments 1-31, wherein the reporter is detected after about any one or more of 1, 2, 3, 4, 5, 6, 7, 8, 12, 16, 20, 24 hours or greater than 24 hours after contacting the antigen-polypeptide complex with the phagocytic cell.

33. A kit for determining the potency of a polypeptide preparation wherein the polypeptide binds a target antigen and comprises an Fc receptor binding domain, the kit comprising an immobilized target antigen and a phagocytic cell, wherein the phagocytic cell comprises an Fcγ receptor and nucleic acid encoding a reporter operably linked to a response element that is responsive to activation by the Fcγ receptor,

wherein expression of the reporter indicates potency of the polypeptide.

34. A kit for quantitating the potency of a polypeptide preparation wherein the polypeptide binds a target antigen and comprises an Fc receptor binding domain, the kit comprising an immobilized target antigen, a phagocytic cell, and a reference standard;

wherein the phagocytic cell comprises an Fcγ receptor and nucleic acid encoding a reporter operably linked to a response element that is responsive to activation by the Fcγ receptor, wherein expression of the reporter indicates potency of the polypeptide; and

wherein the reference standard comprises a preparation of the polypeptide of known potency.

35. The kit of embodiment 33 or 34, wherein the reporter is a luciferase or a fluorescent protein.

36. The kit of embodiment 35, wherein the luciferase is a firefly luciferase, a Renilla luciferase, or a nanoluciferase.

37. The kit of any one of embodiments 33-36, wherein the kit further comprises reagents to detect expression of the reporter.

38. The kit of any one of embodiments 33-37, wherein the response element that is responsive to activation by the Fcγ receptor is an NFκB response element, an NFAT response element, an AP-1 response element, or an ERK-responsive transcription factor (e.g. Elk1).

39. The kit of any one of embodiments 33-38, wherein the phagocytic cell is from a cell line.

41. The kit of embodiment 39, wherein the cell line is a THP-1 cell line or a U-937 cell line.

41. The kit of any one of embodiments 33-40, wherein the Fcγ receptor is a FcγRI (CD64), a FcγRIIa (CD32a) or a FcγRIII (CD16).

42. The kit of any one of embodiments 33-41, wherein the phagocytic cell is engineered to overexpress a Fcγ receptor.

43. The kit of embodiment 42, wherein the phagocytic cell is engineered to overexpress a FcγRIIa.

44. The kit of any one of embodiments 33-43, wherein the phagocytic cell does not express FcγRIII.

45. The kit of any one of embodiments 33-44, wherein the target antigen is beta-amyloid (Aβ) or CD20.

46. The kit of any one of embodiments 33-45, wherein the target antigen is beta-amyloid (Aβ).

47. The kit of embodiment 46, wherein the Aβ is human Aβ.

48. The kit of embodiment 46 or 47, wherein the Aβ comprises monomeric and/or oligomeric Aft

49. The kit of embodiment 48, wherein the human Aβ is Aβ 1-40 or Aβ 1-42.

50. The kit of any one of embodiments 33-49, wherein the polypeptide comprises a full length Fc domain or an FcR-binding fragment of an Fc domain.

51. The kit of any one of embodiments 33-50, wherein the polypeptide specifically binds Aβ.

52. The kit of any one of embodiments 33-51, wherein the polypeptide is an antibody or an immunoadhesin.

53. The kit of embodiment 52, wherein the polypeptide in crenezumab.

54. The kit of any one of embodiments 33-53, wherein the target antigen is immobilized on a surface.

55. The kit of embodiment 54, wherein the surface is a plate.

56. The kit of embodiment 55, wherein the plate is a multi-well plate.

57. The method of embodiment 55 or 56, wherein the target antigen is bound to biotin at or near its N-terminus, at or near its C-terminus, or at or near its N-terminus and at or near its C-terminus.

58. The kit of any one of embodiments 54-57, wherein the target antigen is immobilized on the surface using a biotin-streptavidin system.

59. The kit of embodiment 58, wherein the target antigen is bound to biotin and the surface comprises bound streptavidin.

All of the features disclosed in this specification may be combined in any combination Each feature disclosed in this specification may be replaced by an alternative feature serving the same, equivalent, or similar purpose. Thus, unless expressly stated otherwise, each feature disclosed is only an example of a generic series of equivalent or similar features.

Further details of the invention are illustrated by the following non-limiting Examples. The disclosures of all references in the specification are expressly incorporated herein by reference.

### EXAMPLES

The examples below are intended to be purely exemplary of the invention and should therefore not be considered to limit the invention in any way. The following examples and detailed description are offered by way of illustration and not by way of limitation.

### Example 1

### Materials and Methods

### Phagocytosis Reporter Cell Generation

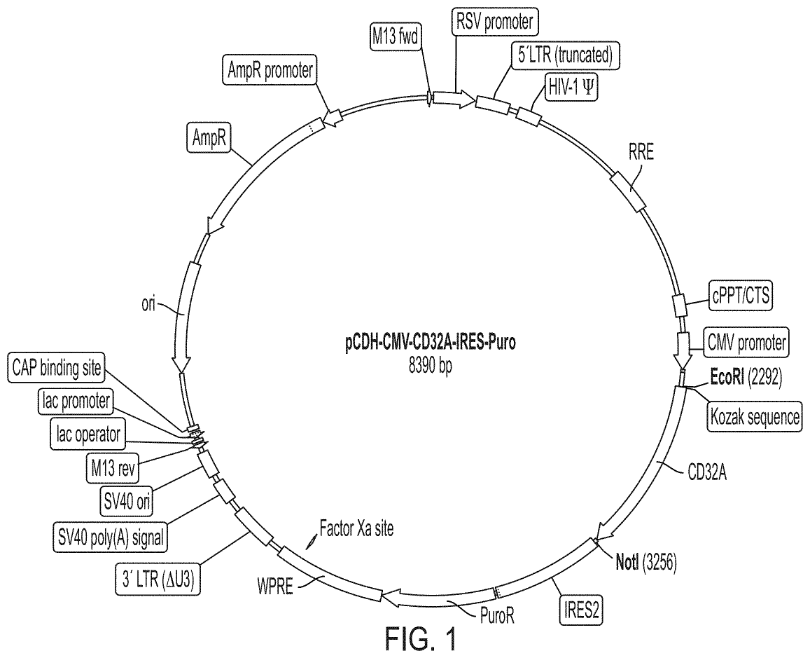

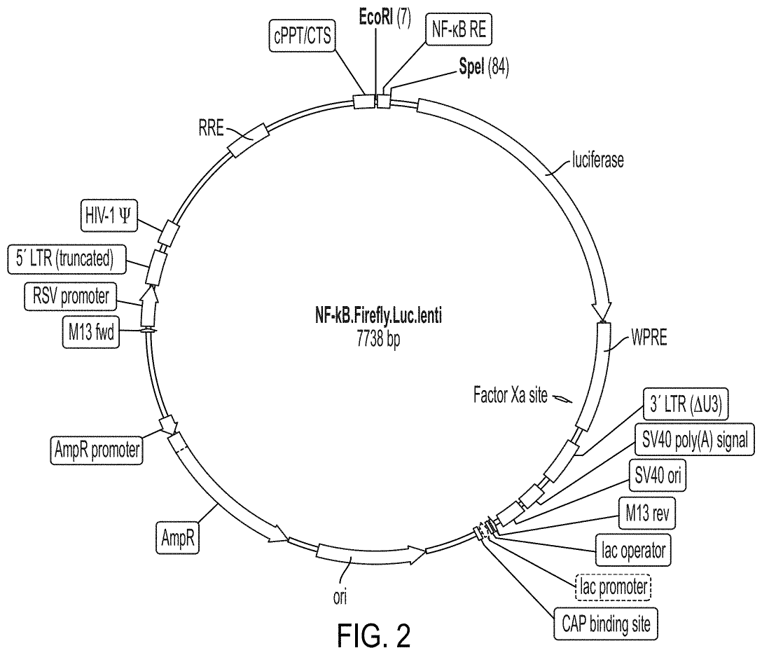

Human FCGR2A (CD32A) cDNA (protein_id=NP_067674.2; coded by=NM_021642.3; HIS variant) was first chemically synthesized (GeneArt™ Gene Synthesis). Restriction sites (EcoRI, 5′ end; NotI, 3′ end) were added to the cDNA template as well as a Kozak sequence (GCCACC) immediately 5′ of the ATG start codon. The cDNA was subcloned into lentiviral vector pCDH-CMV-MCS-IRES-Puro using EcoRI and NotI (FIG. 1). The resulting construct, pCDH-CMV-CD32A-IRES-Puro was sequenced to confirm the entire cDNA insert. A reporter construct was generated by cloning a nuclear factor-κB (NF-κB) response element (RE) into a lentiviral vector upstream of the firefly luciferase (Luc) gene (FIG. 2). Lentivirus particles were generated using these constructs, and these were used to transduce U-937 and THP-1 monocyte cell lines. Parental pools were generated by selection with 1 μg/mL puromycin (Clontech), and luminescence activity was confirmed using TNFα, which also activates NF-κB, as a positive control. Limiting dilution was performed to isolate clones, and clones were screened for activity with crenezumab and amyloid β (Aβ). Cells were cultured in RPMI (Gibco) with 10% heat-inactivated fetal bovine serum (HI FBS) (Gibco), 1× Glutamax (Gibco), and 1× Penicillin-Streptomycin (Gibco), and frozen in 90% HI FBS, 10% dimethyl sulfoxide (DMSO) (ATCC).

### Reagents and Buffers

Non-biotinylated Aβ peptide (rPeptide or Anaspec) and biotin beta-amyloid 1-42 peptide (Biotin-Aβ) (Anaspec) was reconstituted by first adding 40 μL room temperature DMSO to each 0.5 mg vial of the peptide. The walls of the vial were washed 2-3 times followed by addition of 960 μL of phosphate-buffered saline (PBS) adjusted to pH 8.0. Vials were vortexed for approximately 1 min until reagent was dissolved, then reagent was pooled, aliquoted, and stored at ≤−60° C. until use. An additional peptide included a 51-amino acid peptide of CD20 with biotin each end (CD20-biotin) (CPC Scientific). This peptide was similarly reconstituted in DMSO and brought to a stock concentration of 1 mg/mL with PBS.

TBS Binding Buffer consists of Tris-Buffered Saline (10 mM Tris pH 8.0, 150 mM NaCl). Wash Buffer consists of PBS with 1 mM CaCl₂, 1 mM MgCl₂. Assay Medium is RPMI (Gibco) with 10% HI-FBS (Gibco), 1× Glutamax (Gibco), and 1× Penicillin-Streptomycin (Gibco). In early development and for the ocrelizumab version of the assay, Low-IgG HI FBS (Hyclone Ultra-Low IgG or Gibco) was used as an alternative to HI-FBS. Quantitation of luciferase expression utilizes a luminescent reagent (Promega, Steady-Glo® Luciferase Assay System). ELISA Block Buffer was Dulbccco's phosphate-buffered saline (DPBS) with 1 mM CaCl₂) and 1 mM MgCl₂ plus 0.5% bovine serum albumin (BSA). ELISA Assay Diluent was PBS, 0.5% BSA. 0.05% polysorbate 20.

Crenezumab reference standard and samples were manufactured by Genentech. Formulation Buffer is 200 mM arginine succinate, 0.05% (w/v), polysorbate 20, pH 5.5±0.3. To generate the light stress samples, 25 mL crenezumab was placed in a glass vial and set in a calibrated light box for a cumulative exposure of 2.4 million lux hours over a period of 16 hours; light control was wrapped in aluminum foil for the exposure.

### Flow Cytometry

Cells were washed in PBS or FACS Wash (0.5% bovine serum albumin, 0.1% sodium azide in PBS) and resuspended in FACS Wash. For the U-937 experiment, cells were first stained with a vital dye (Invitrogen) and preincubated with Fe blocking antibodies (eBioscience, anti-CD16: 16-0166-85, anti-CD32: 16-0329-85, anti-CD64: 14-0649-82) for 10-15 min. Cells were then stained with the following anti-FcγR antibodies or isotype controls for 30-60 minutes: CD16-phycoerythrin (PE) (eBio, 12-0167-42), CD32-PE (BD Pharmingen, 550586), CD64-PE (eBio, 12-0649), CD64-FITC (eBio, 11-0649-42), FITC-mouse IgG1κ (eBio, 11-4714-42), PE-mouse IgG1κ (eBio, 12-4714-42), PE-mouse IgG2bκ (BD Pharmingen, 555743). Cells were washed and resuspended in FACS Wash, and fluorescence was detected on a flow cytometer (BD, LSR II or FACSCaliber).

Evaluation of Aβ Peptide and Plate Format

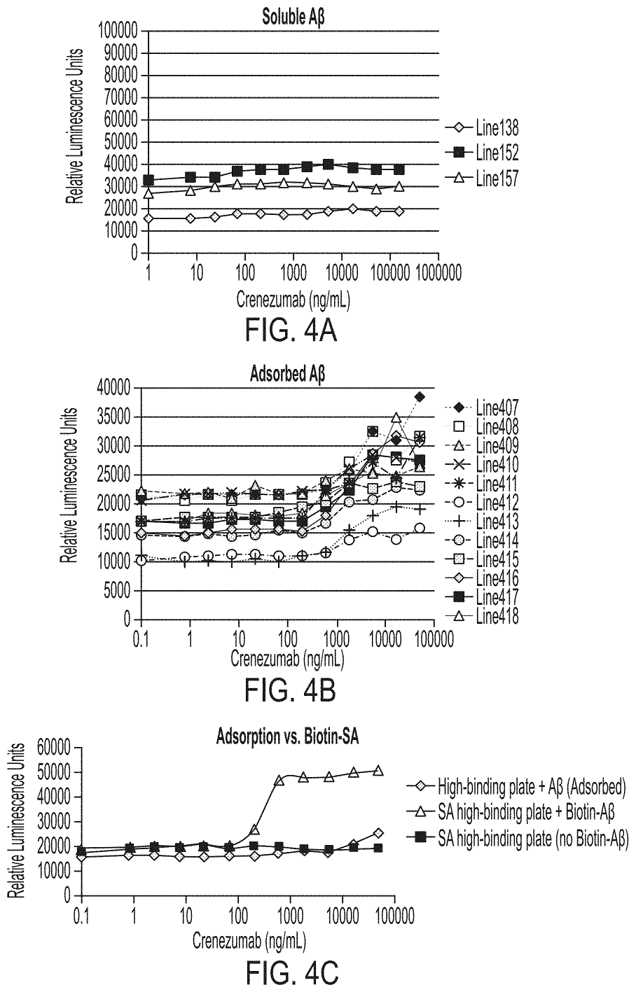

Soluble, non-biotinylated Aβ at 5 μg/mL (25 μL) was incubated with a ⅓ dilution series of crenezumab (25 μL, starting concentration 600,000 ng/mL) and THP-1 phagocytosis reporter cells (50 μL, 500,000 cells/mL) in assay medium in a white tissue culture-treated assay plate (Costar) for 5 hours at 37° C. Luminescent reagent Steady-Glo® (100 μL) (Promega) was added, shaking for 20 min, and luminescence was detected using a luminescence plate reader (Perkin-Elmer, EnVision). Alternatively, 100 μL non-biotinylated Aβ at 1 μg/mL in PBS was adsorbed onto a high-binding white plate (Thermo, Maxisorp) overnight at 4° C. Plates were washed with PBS, blocked with 200 μL assay medium for 30 min, and washed again. Then, plates were incubated with 100 μL of a ⅓ dilution series of crenezumab in assay medium (starting concentration 50,000 ng/mL) at 37° C. for 30 min. Plates were washed again, and 100 μL of THP-1 phagocytosis reporter cells at 200,000 cells/mL were incubated for 5 hours at 37° C. Luminescent reagent Steady-Glo® (100 μL) (Promega) was added, shaking for 20 min, and luminescence was detected using a luminescent plate reader (Perkin-Elmer, EnVision). This variation on the procedure was also used to first evaluate Biotin-Aβ and streptavidin high binding capacity 96-well white plates (FIG. 4).

### Crenezumab Aβ Binding ELISA

Recombinant human amyloid β 1-42 peptide (rPeptide) was reconstituted in DMSO and frozen in single-use aliquots. For the assay, the peptide was diluted to 1 μg/mL in DPBS, and 100 μL was added to a high-binding polystyrene plate (Nunc). Plates were incubated for 16-72 hours at 2-8° C., then dumped and blocked with 200 μL ELISA Block Buffer for 1-2 hours at 25° C. Plates were washed with PBS+0.05% polysorbate 20, and 100 μL crenezumab reference standard and sample dilutions in ELISA Assay Diluent were added. Plates were incubated at 25° C. for 1 hour then washed again. A 2 ng/mL solution of goat anti-human IgG-horseradish peroxidase (HRP) (Jackson Immunoresearch) was added to the plate for 40 min at 25° C. before washing A colorimetric TMB detection reagent (SureBlue Reserve, KPL) was added, and plates were developed, shaking, for 10-30 min prior to the addition of 0.6 N sulfuric acid. Absorbance was detected at 450 nm using a plate reader (Molecular Devices), and absorbance at 650 nm was used as a reference absorbance. Potency relative to crenezumab reference standard was calculated using a parallel line analysis curve-fitting program.

### Crenezumab Phagocytosis Reporter Method

Biotin-Aβ was diluted to a concentration of 1.5 μg/mL in TBS Binding Buffer and bound to a Streptavidin High Binding Capacity Coated 96-well white plate (Pierce, Thermo Scientific) for 16-72 hours at 25° C. Plates were washed three times with Wash Buffer using a plate washer (Biotek) and equilibrated with warm Assay Medium for 1-2.5 hours at 37° C. in a humidified incubator with 5% CO2, covered with a breathable plate sealer (Aeraseal, Sigma) or lid. Reference standard and samples were diluted in Formulation Buffer for protein quantitation by UV SpecScan. An 8-point dilution curve was prepared for reference standard, assay control (independent dilution of reference standard), and samples in warm Assay Medium targeting concentrations of 10,000, 4000, 1600, 750, 250, 100, 40, and 10 ng/mL. Phagocytosis reporter cells were harvested from flasks by centrifugation, resuspended in warm Assay Medium, counted, and diluted to 2.5×10⁶ cells/mL. Plates were washed again, and 50 μL each of sample dilution and cell preparation was added. Plates were incubated for 3-5 hours at 37° C. in a humidified incubator with 5% CO2, covered with a breathable plate sealer or lid. Assay plates were then cooled in a 25° C. incubator for 15-20 min followed by addition of 100 μL luminescent reagent. Plates were shaken at room temperature using a table-top shaker, then luminescence signal is detected on a luminescence plate reader (Molecular Devices, Paradigm or i3x equipped with a LUM96 cartridge). Potency was calculated based on the EC50 ratio using a 4P constrained fit against the crenezumab reference standard. Plate reads and potency calculations were performed using software (Molecular Devices, SoftMax® Pro v6.5).

### Ocrelizumab Phagocytosis Reporter Method

The ocrelizumab test method was similar to the crenezumab test method with the following modifications. CD20-biotin peptide was diluted to 8 μg/mL in PBS, pH 6.5 for binding to the plate at 2-8° C. for 16-72 hours. Wash Buffer was PBS+0.05% polysorbate 20. Following peptide binding, plates were washed six times, equilibrated with Assay Medium, washed, and incubated with 100 μL dilutions of ocrelizumab for 1.5 hours at 37° C. Ocrelizumab concentrations were 100,000, 30,000, 15,000, 8000, 4000, 2000, 1000, and 100 ng/mL. Plates were then washed, and 100 μL of U-937 phagocytosis reporter cells at a concentration of 1.5×10⁶ cells/mL were added. Plates were incubated for 2 hours 40 minutes at 37° C. Low IgG HI FBS was used in the Assay Medium.

### Results

### Phagocytosis Reporter Cells

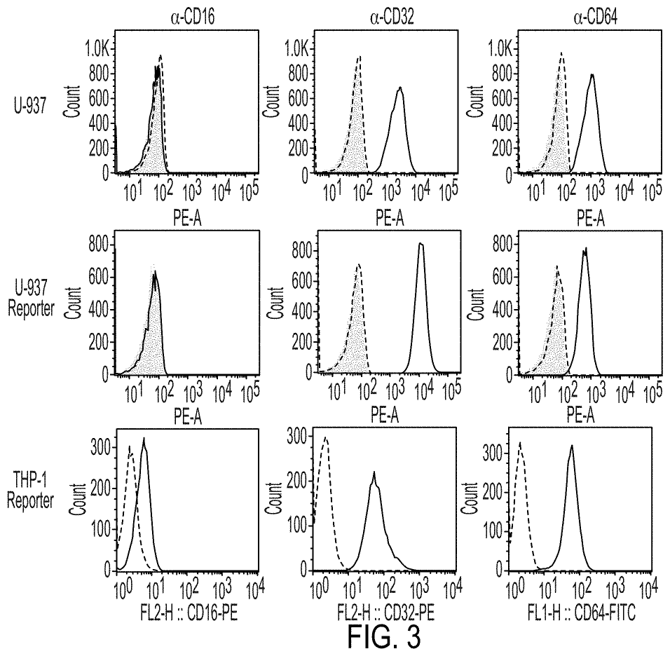

The phagocytosis reporter cell assay was first developed for crenezumab, which binds to soluble Aβ oligomers and facilitates uptake of immune complexes by microglia (Adolfsson et al.). This mechanism is analogous to antibody-dependent cellular phagocytosis (ADCP) in that it involves phagocytic cells and is mediated by Fcγ receptors (FcγRs). To best reflect the biology of ADCP, phagocytic human monocyte cell lines, THP-1 and U-937, were selected as the parental cell lines to generate the phagocytosis reporter cell line. THP-1 and U-937 cell lines were engineered to express a firefly luciferase gene under the control of an NF-κB response element as described in Materials and Methods. NF-κB is a transcriptional regulator induced by signaling through FcγRs, among other immune receptors. While the specific Fcγ receptor(s) involved microglial clearance of Aβ by crenezumab is unknown, crenezumab, an IgG4, binds with highest affinity to CD64 (FcγRI). CD32A (also known as FcγRIIa) is considered to be relevant to ADCP due to its preference for immune complexes over monomeric IgG, and this receptor is also sensitive to Fc galactosylation, a potential product variant for antibody therapeutics. Therefore, cells were engineered with an additional CD32A construct to maximize sensitivity to potential product variants. U-937 and THP-1 cells also express CD64, but low to no CD16 (FcγRIIIa) (FIG. 3). Both the U-937 and THP-1 reporter cells are representative of the phagocytosis mode of action, and a potency assay was optimized, including selection of cell line, for each specific antibody and target. THP-1 cells were ultimately selected for the crenezumab potency assay due to better assay precision and consistency for this antibody/target.

### Evaluation of Aβ Peptide and Plate Format

Three approaches were evaluated to introduce Aβ peptide oligomers into the assay (FIG. 4). The first utilized soluble Aβ peptide preparations, which may form oligomers in aqueous solutions, mixed with crenezumab and reporter cells. This approach failed to yield a luminescence signal, possibly due to incomplete or inefficient formation of Aβ oligomeric complexes. To mimic Aβ complexes and/or seed the formation of complexes, plate-bound formats were explored in which crenezumab and reporter cells were layered onto plates coated with Aβ peptide. Aβ peptide adsorbed onto high-binding plates showed a positive, but inconsistent, signal in the reporter cells. To improve signal and the consistency of Aβ binding to the plate surface, a streptavidin (SA)-biotin system was utilized in which biotinylated Aβ is bound to streptavidin-coated plates.

### Assay Format and Crenezumab Standard Curve

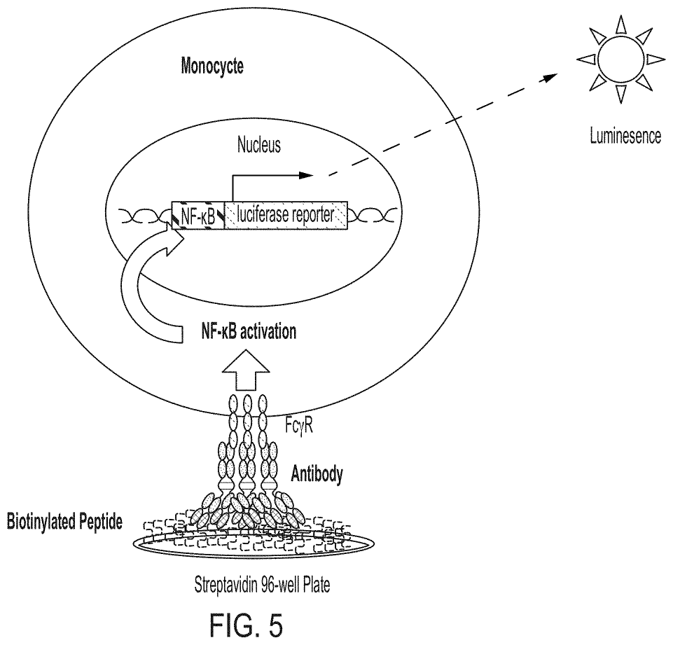

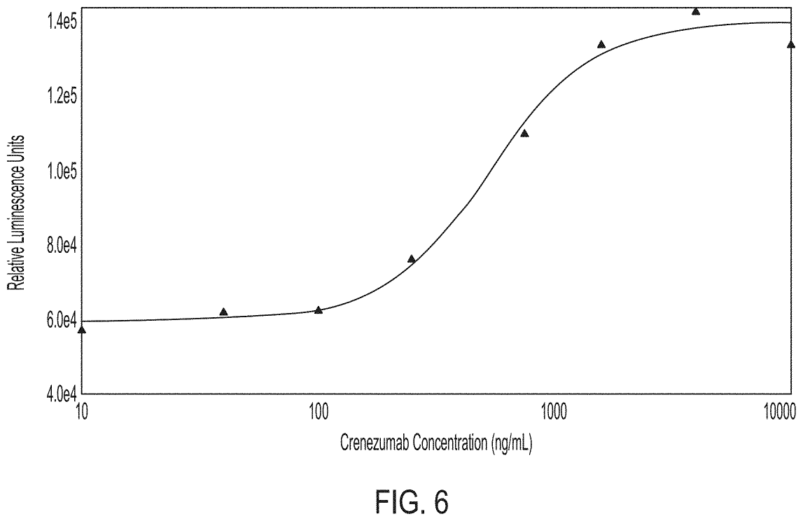

The format of the phagocytosis reporter cell assay involves binding of a biotinylated peptide onto a streptavidin-coated plate (FIG. 5). Peptide-specific antibody binds to the peptide target and triggers clustering and activation of FcγRs. This leads to activation of NF-κB and expression of the reporter gene, namely luciferase, which allows quantitation of luminescence upon addition of a substrate. A representative dose response curve for crenezumab reference standard is shown in FIG. 6.

### Example of Crenezumab Potency Determination: Degraded Samples

The assay was used to determine the potency of crenezumab samples. To demonstrate that the phagocytosis reporter cell assay can detect changes in potency due to product degradation, crenezumab stress samples from a light stress study were tested for activity. These samples exhibited a loss in Aβ binding activity as measured by an ELISA, and a similar loss in potency was observed using the phagocytosis reporter cell assay (Table 2), demonstrating that the reporter cell assay can detect potency loss caused by a loss in Aβ binding activity.

TABLE 2
Potency of crenezumab light stress samples

| | Aβ Binding | Phagocytosis Reporter |
|---|---|---|
| Sample | Potency | Cell Potency |
| Light Control | 107 | 99 |
| Light Stress | 84 | 86 |
| 2.4 M lux h | | |

Results are % Relative Potency assigning crenezumab reference standard as 100% and are the mean of three independent assays.

### Application of Assay Format to Other Products/Targets

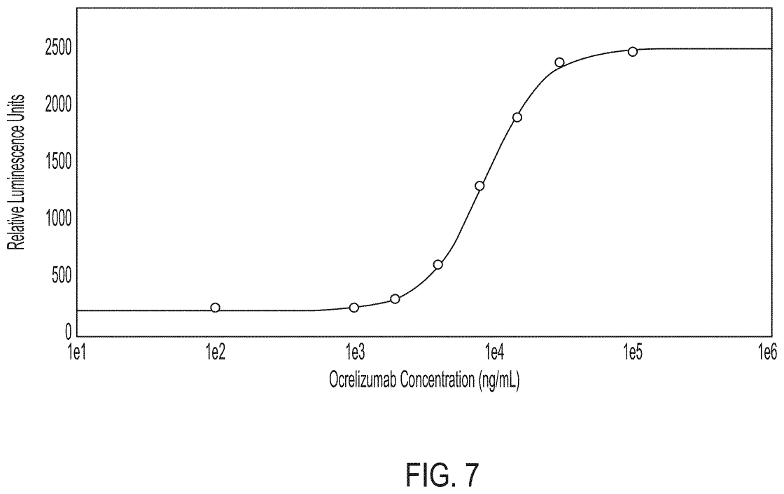

To determine if the phagocytosis reporter assay could be applied to other antibody products, the format was adapted to other peptide target/antibody combinations. Ocrelizumab is a CD20-binding antibody with ADCP as a proposed mechanism of action. Therefore, a biotinylated CD20 peptide was bound to the streptavidin plate, and ocrelizumab was bound to the peptide to mimic the binding of ocrelizumab to the surface of a CD20-expressing cell. Using U-937 phagocytosis reporter cells, a luminescent signal was observed to generate a dose response curve. This allows the assessment of ADCP potency for ocrelizumab (FIG. 7).

### SUMMARY

An assay was developed to measure the potency of crenezumab using a reporter cell line and plate-bound peptide (FIG. 5). The assay functions as a surrogate for FcγR-mediated uptake of immune complexes/ADCP. It is reflective of the mode of action in that it utilizes a phagocytic monocyte cell line and measures engagement and activation of FcγRs by immune complexes of crenezumab and Aβ peptide. The assay was demonstrated to be sensitive to losses in potency using crenezumab stress samples. Furthermore, the assay format can be applied to other targets and products as demonstrated for ocrelizumab (CD20 binding).

### Example 2

The development of the phagocytosis reporter cells and assay format were described above. Here, additional data regarding optimization of assay conditions for the crenezumab assay are described.

### Cell Line Optimization

THP-1 and U-937 cell lines were engineered to express a firefly luciferase gene under the control of a nuclear factor-(NF-κB) response element to overexpress CD32 to maximize sensitivity to potential product variants.

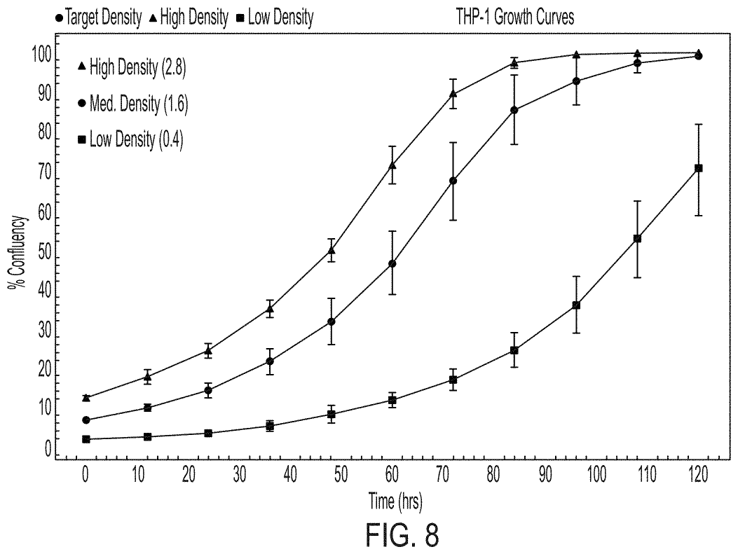

The engineered U-937 cell line was initially selected for the crenezumab assay based on higher fold responses and faster growth. However, following additional comparisons of crenezumab assay performance, THP-1 phagocytosis reporter cells were selected. An experiment was performed to assess impact of cell seeding density on growth for THP-1 phagocytosis reporter cells to improve cell growth and yield for the assay. THP-1 cells grew slower at lower cell seeding densities (FIG. 8), therefore relatively high seeding concentrations were incorporated into the cell culture procedure.

### Selection and Optimization of Reagents

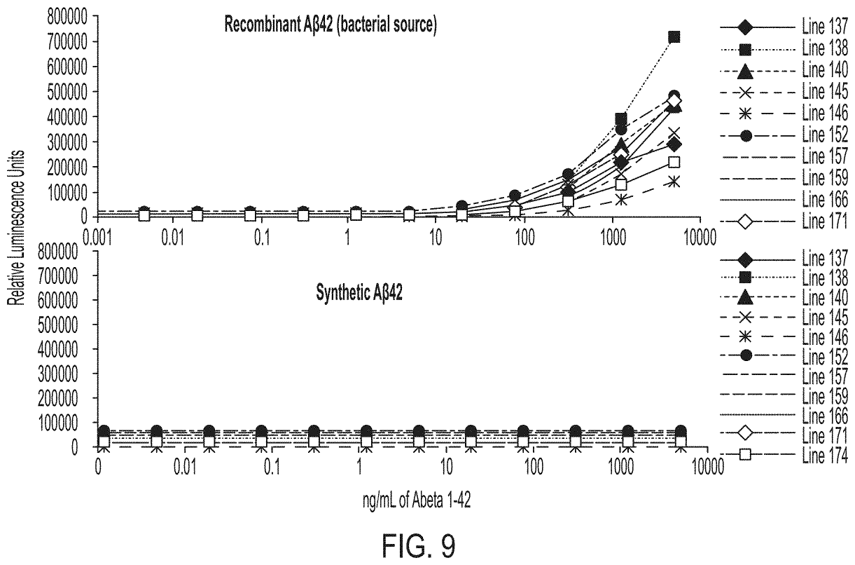

NF-κB is activated downstream of several immune receptors, so a potential concern was off-target activation of reporter cells by contaminants, such as bacterial lipopolysaccharide (LPS) in recombinant Aβ peptide preparations. Therefore, Aβ peptides from recombinant and synthetic sources were compared for their ability to activate reporter cells in the absence of crenezumab (FIG. 9). Synthetic Aβ peptide was selected to minimize potential for endotoxin-mediated activation of reporter cells.

### Design of Experiment Optimization of Assay Parameters

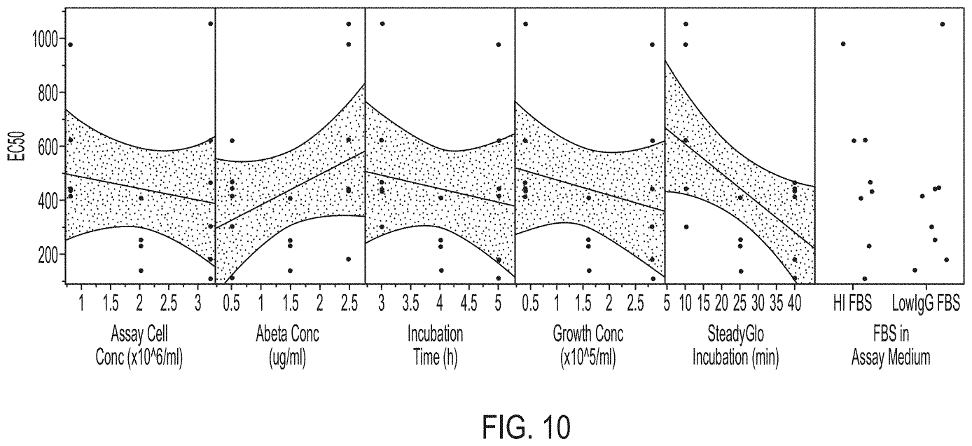

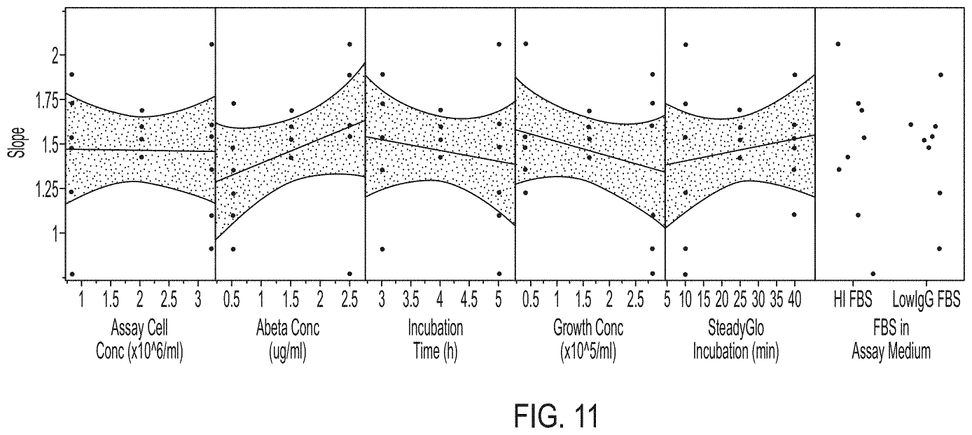

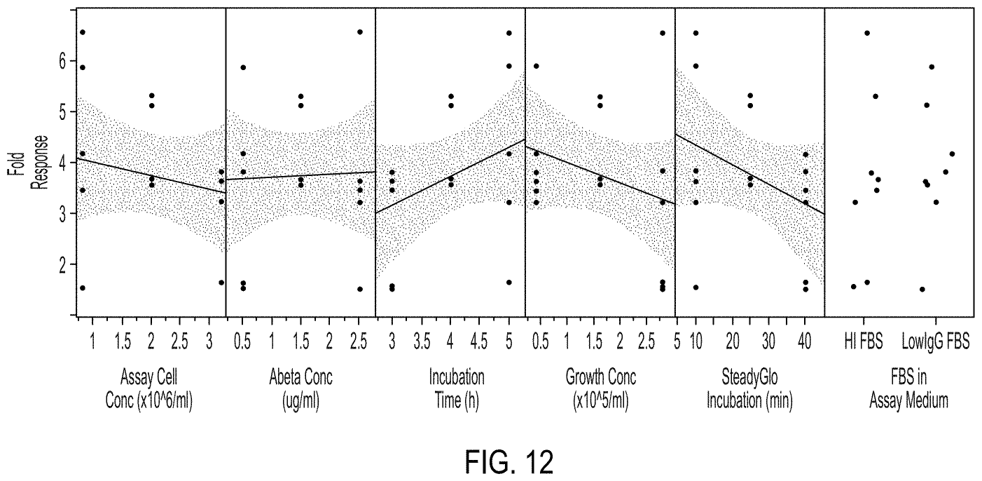

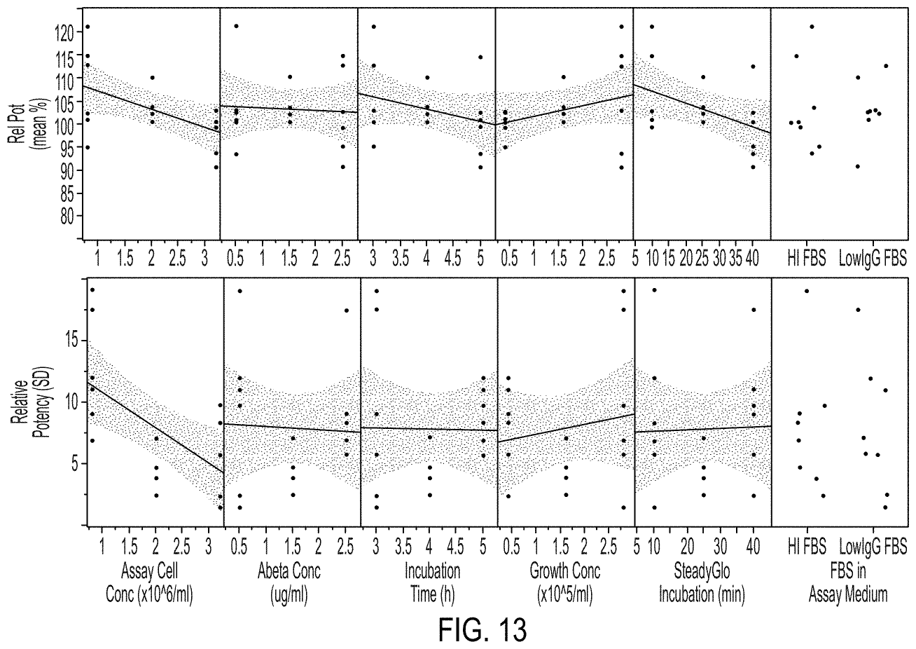

To further optimize the assay and evaluate impact of assay factors on assay readouts, a Plackett-Burman design was used. The assay factors evaluated included assay cell concentration, Aβ peptide concentration, incubation time, cell growth concentration (seeding density in flask), SteadyGlo® incubation time, and type of FBS (HI vs. low IgG FBS) (Table 3). Additionally, two batches of Aβ peptide preparation and multiple analysts were incorporated into the design. Assay factors were evaluated for their impact on EC₅₀, slope, fold response, potency mean, and potency standard deviation (SD) in a main effects analysis (FIGS. 10 to 13).

TABLE 3

| | | | | Cell Growth | | | | |
|---|---|---|---|---|---|---|---|---|
| | Assay Cell | | | Seeding | SteadyGlo | FBS in | | |
| | Concentration | Aβ Conc. | Incubation | Density | Incubation | Assay | Aβ | Analyst/ |
| # | (×10 [symbol] /mL) | (μg/mL) | Time (h) | (×10⁵/mL) | (min) | Medium | Batch | Group |
| 1 | 2 | 1.5 | 4 | 1.6 | 25 | Hl | 2 | 1 |
| 2 | 3.2 | 2.5 | 5 | 0.4 | 10 | Hl | 2 | 1 |
| 3 | 0.8 | 2.5 | 3 | 0.4 | 40 | Hl | 2 | 2 |
| 4 | 3.2 | 2.5 | 3 | 0.4 | 10 | LowlgG | 1 | 1 |
| 5 | 0.8 | 0.5 | 5 | 0.4 | 10 | LowlgG | 1 | 2 |
| 6 | 2 | 1.5 | 4 | 1.6 | 25 | LowlgG | 1 | 2 |
| 7 | 0.8 | 0.5 | 5 | 0.4 | 40 | LowlgG | 2 | 1 |
| 8 | 0.8 | 2.5 | 5 | 2.8 | 10 | Hl | 1 | 2 |
| 9 | 0.8 | 2.5 | 3 | 2.8 | 40 | LowlgG | 1 | 1 |
| 10 | 0.8 | 0.5 | 3 | 2.8 | 10 | Hl | 2 | 1 |
| 11 | 2 | 1.5 | 4 | 1.6 | 25 | Hl | 1 | 2 |
| 12 | 3.2 | 0.5 | 3 | 2.8 | 10 | LowlgG | 2 | 2 |
| 13 | 3.2 | 0.5 | 5 | 2.8 | 40 | Hl | 1 | 1 |
| 14 | 3.2 | 2.5 | 5 | 2.8 | 40 | LowlgG | 2 | 2 |
| 15 | 3.2 | 0.5 | 3 | 0.4 | 40 | Hl | 1 | 2 |
| 16 | 2 | 1.5 | 4 | 1.6 | 25 | LowlgG | 2 | 1 |

[symbol]

indicates data missing or illegible when filed

1. A method for determining the activity of a polypeptide wherein the polypeptide binds a target antigen and the polypeptide comprises an Fc receptor binding domain, the method comprising
a) contacting an immobilized target antigen with the polypeptide preparation to form an antigen-polypeptide complex,
b) contacting the antigen-polypeptide complex with a phagocytic cell, wherein the phagocytic cell comprises an Fcγ receptor and nucleic acid encoding a reporter operably linked to a response element that is responsive to activation by the Fcγ receptor;
wherein expression of the reporter indicates activity of the polypeptide.

2. A method for quantitating the potency of a polypeptide preparation wherein the polypeptide binds a target antigen, the method comprising
a) contacting a plurality of populations of immobilized target antigen with different concentrations of the polypeptide preparation to form antigen-polypeptide complexes,
b) contacting the antigen-polypeptide complexes with a phagocytic cell, wherein the phagocytic cell comprises an Fcγ receptor and nucleic acid encoding a reporter operably linked to a response element that is responsive to activation by the Fcγ receptor,
c) measuring expression of reporter, and
d) determining the EC₅₀ of the polypeptide preparation and comparing the EC₅₀ of the polypeptide preparation with the EC₅₀ of a reference standard of the polypeptide of known potency.

3. The method of claim 2, further comprising calculating the potency based on the EC₅₀ of the polypeptide preparation using a multi-parameter logistic fit against the reference standard.

4. The method of claim 3, wherein the multi-parameter logistic fit is a 3-parameter, 4-parameter, or 5-parameter logistic fit.

5. The method of claim 2, wherein the EC₅₀ of the reference standard is determined at the same time as the EC₅₀ of the polypeptide preparation.

6. The method of claim 1, wherein the reporter is a luciferase or a fluorescent protein.

7. The method of claim 6, wherein the luciferase is a firefly luciferase, a Renilla luciferase, or a nanoluciferase.

8. The method of claim 1, wherein the response element that is responsive to activation by the Fcγ receptor is an NFκB response element, an NFAT response element, an AP-1 response element, or an ERK-responsive transcription factor.

9. The method of claim 1, wherein the phagocytic cell is a monocyte.

10. The method of claim 1, wherein the phagocytic cell is from a cell line.

11. The method of claim 10, wherein the cell line is a THP-1 cell line or a U-937 cell line.

12. The method of claim 1, wherein the Fcγ receptor is a FcγRI (CD64) or FcγRIIa (CD32a) or FcγRIII (CD16).

13. The method of claim 1, wherein the phagocytic cell is engineered to overexpress a Fcγ receptor.

14. The method of claim 13, wherein the phagocytic cell is engineered to overexpress a FcγRIIa.

15. The method of claim 1, wherein the phagocytic cell does not express FcγRIII.

16. The method of claim 1, wherein the target antigen is beta-amyloid (Aβ) or CD20.

17. (canceled)

18. The method of claim 16, wherein the target antigen is human A3.

19. The method of claim 18, wherein the Aβ comprises monomeric and/or oligomeric Aβ.

20. The method of claim 18, wherein the human Aβ is Aβ 1-40 or Aβ 1-42.

21. The method of claim 1, wherein the polypeptide comprises a full length Fc domain or an FcR-binding fragment of an Fc domain.

22. The method of claim 1, wherein the polypeptide specifically binds Aβ.

23. The method of claim 1, wherein the polypeptide is an antibody or an immunoadhesin.

24. The method of claim 22, wherein the polypeptide in crenezumab.

25. The method of claim 1, wherein the target antigen is immobilized on a surface.

26. The method of claim 25, wherein the surface is a plate.

27. The method of claim 26, wherein the plate is a multi-well plate.

28. The method of claim 25 any one of claims 25-27, wherein the antigen is immobilized to the surface at or near its N-terminus, at or near its C-terminus, or at or near its N-terminus and at or near its C-terminus.

29. The method of claim 25, wherein the target antigen is immobilized on the surface using a biotin-streptavidin system.

30. The method of claim 29, wherein the target antigen is bound to biotin and the surface comprises bound streptavidin.

31. The method of claim 29, wherein the target antigen is bound to biotin at or near its N-terminus, at or near its C-terminus, or at or near its N-terminus and its C-terminus.

32. The method of claim 1, wherein the reporter is detected after about any one or more of 1, 2, 3, 4, 5, 6, 7, 8, 12, 16, 20, 24 hours or greater than 24 hours after contacting the antigen-polypeptide complex with the phagocytic cell.

33. A kit for determining the potency of a polypeptide preparation wherein the polypeptide binds a target antigen and comprises an Fc receptor binding domain, the kit comprising an immobilized target antigen and a phagocytic cell, wherein the phagocytic cell comprises an Fcγ receptor and nucleic acid encoding a reporter operably linked to a response element that is responsive to activation by the Fcγ receptor,
wherein expression of the reporter indicates potency of the polypeptide.

34. A kit for quantitating the potency of an polypeptide preparation wherein the polypeptide binds a target antigen and comprises an Fc receptor binding domain, the kit comprising an immobilized target antigen, a phagocytic cell, and a reference standard;
wherein the phagocytic cell comprises an Fcγ receptor and nucleic acid encoding a reporter operably linked to a response element that is responsive to activation by the Fcγ receptor, wherein expression of the reporter indicates potency of the polypeptide; and
wherein the reference standard comprises a preparation of the polypeptide of known potency.

35-59. (canceled)


SEQUENCE LISTING (2 sequences)

SEQ ID NO: 1 - protein, 42 aa, Homo sapiens [sequence omitted]

SEQ ID NO: 2 - protein, 40 aa, Homo sapiens
DAEFRHDSGYEVHHQKLVFFAEDVGSNKGAIIGLMVGGVV